(19) United States
(12) Patent Application Publication
(10) Pub. No.: US 2021/0128755 A1
(43) Pub. Date: May 6, 2021

(54) TARGETING LIGANDS FOR TAU PATHOLOGY

(71) Applicants: Alzeca Biosciences, LLC, Houston, TX (US); Texas Children's Hospital, Houston, TX (US)

(72) Inventors: Ananth Annapragada, Manvel, TX (US); Qingshan Mu, North Andover, MA (US); Carlo Medici, Reno, NV (US)

(73) Assignees: Alzeca Biosciences, LLC, Houston, TX (US); Texas Children's Hospital, Houston, TX (US); Baylor College of Medicine, Houston, TX (US)

(21) Appl. No.: 16/922,762

(22) Filed: Jul. 7, 2020

Related U.S. Application Data
(60) Provisional application No. 62/871,380, filed on Jul. 8, 2019.

(51) Int. Cl. A61K 49/18; A61K 49/08; C12N 15/115

(52) U.S. Cl. CPC A61K 49/1812; C12N 2310/16; C12N 15/115; A61K 49/085

(57) ABSTRACT

Methods and compositions for detecting tau pathology are described. The compositions for detecting tau pathology comprise a targeting ligand that specifically binds to a cell surface marker of tau pathology, wherein the targeting ligand is linked to a liposome that includes an imaging agent. The compositions can be used in a method for imaging tau pathology in a subject that comprises administering to the subject an effective amount of the composition to a subject and imaging at least a portion of the subject to determine if that portion of the subject exhibits tau pathology. The compositions can also be used to detect tau pathology in biological samples obtained from a subject.

### CROSS-REFERENCE TO RELATED APPLICATIONS

This application claims priority from U.S. Provisional Patent Application No. 62/871,380, filed on Jul. 8, 2019, which is incorporated by reference herein in its entirety.

### SEQUENCE LISTING

A Sequence Listing has been submitted electronically in ASCII format and is hereby incorporated by reference in its entirety. The ASCII copy, created on Jul. 7, 2020, is named Alzeca-121 Sequence Listing ST25.txt and is 47,652 bytes in size.

### BACKGROUND

The microtubule associated protein tau is integral to the pathogenesis of Alzheimer's Disease (AD) and other tauopathies. Tau is coded for by the MAPT gene. Alternate splicing generates six tau isoforms that differ by the regulated insertion of two inserts close to the N terminus (0N, 1N, 2N) and either three or four repeat sequences (3R, 4R) corresponding to the conserved microtubule binding domain close to the C terminus. The 4R:3R ratio of both mRNA and protein is close to 1:1 in normal brain tissue, but increases in pathological states.

Tau pathology occurs via several molecular changes: phosphorylation, acetylation, ubiquitination, SUMOylation, glycation, nitration, and truncation. However, all of these molecular changes are associated with abnormal phosphorylation, leading to the conclusion that abnormal phosphorylation is the first step in the formation of tau pathology. Abnormal phosphorylation of tau leads to the formation of paired helical filaments constituting the majority of neurofibrillary tangles found in neuronal cells that degenerate during the course of AD. These tangles, in combination with amyloid plaques, constitute the two pathological hallmarks of AD.

A conclusive diagnosis of AD by the most recent criteria supported by the National Institute for Aging and the Alzheimer's Association requires pathological amyloid and tau (A+, T+), with the amyloid being measured by one of the approved PET imaging agents, and the tau being measured by an imaging agent or by cerebrospinal fluid (CSF) levels. The time course of key biomarkers such as amyloid PET and CSF tau as the disease progresses has long been studied. The most recent consensus suggests that pathological tau levels lag pathological amyloid levels by several years. By the time significant elevations in both markers can be detected by conventional means, the disease is typically already in an advanced state. Further, while the identification of amyloid plaques by PET tracers is objective and unequivocal, CSF and blood markers of tau are confounded by numerous other factors, including other pathologies and treatments under which the patient may be going. Artificial Intelligence-interpreted proteomic analyses of serum biomarkers have generated much interest recently, but are still only fractionally accurate against a PET gold standard.

An early indicator of tau pathology may advance diagnosis of AD by several years, perhaps to a pre-symptomatic stage of the disease.

### SUMMARY

A composition for identifying tau pathology is provided, the composition comprising a targeting ligand that specifically binds to a cell surface marker of tau pathology, wherein the targeting ligand is linked to a liposome comprising an imaging agent, e.g., a magnetic resonance imaging (MRI) contrast enhancing agent. In some aspects, the targeting ligand comprises an aptamer or stabilized aptamer. In some aspects, the targeting ligand comprises a thioaptamer. In some aspects, the targeting ligand comprises a DNA nucleotide sequence selected from one or more of Tau_1 (SEQ ID NO: 5), Tau_3 (SEQ ID NO: 6), Tau_9 (SEQ ID NO: 7), Tau_11 (SEQ ID NO: 8), Tau_10 (SEQ ID NO: 9), Tau_13 (SEQ ID NO: 10), Tau_8 (SEQ ID NO: 11), Tau_4 (SEQ ID NO: 12), Tau_17 (SEQ ID NO: 13), Tau_5 (SEQ ID NO: 14), Tau_21 (SEQ ID NO: 15), Tau_25 (SEQ ID NO: 16), Tau_7 (SEQ ID NO: 17), Tau_31 (SEQ ID NO: 18), Tau_42 (SEQ ID NO: 19), Tau_14 (SEQ ID NO: 20), Tau_19 (SEQ ID NO: 21), Tau_15 (SEQ ID NO: 22), Tau_56 (SEQ ID NO: 23), Tau_34 (SEQ ID NO: 24), Tau_23 (SEQ ID NO: 25), Tau_99 (SEQ ID NO: 26), and Tau_102 (SEQ ID NO: 27). In some aspects, the cell surface marker of tau pathology comprises a cell surface marker of tau hyperphosphorylation. In some aspects, the cell surface marker of tau pathology comprises a protein selected from keratin 6A (KRT6A), keratin 6B (KRT6B), heat shock protein (HSP), and vimentin (VIM). In some aspects, the targeting ligand is determined to specifically bind to a cell surface marker of tau pathology using a systematic evolution of ligands by exponential enrichment (SELEX) method. In some aspects, the targeting ligand is linked to polyethylene glycol that is conjugated to a phospholipid that associates with the liposome. In some aspects, the liposome comprises a membrane, the membrane comprising: a first phospholipid; a sterically bulky excipient that is capable of stabilizing the liposome; a second phospholipid that is derivatized with a first polymer; a third phospholipid that is derivatized with a second polymer, the second polymer being conjugated to the targeting ligand; and an imaging agent that is encapsulated by or bound to the membrane.

A method for imaging tau pathology in a subject is also provided, the method comprising: administering to the subject a detectably effective amount of a targeting ligand-liposome conjugate comprising a targeting ligand that specifically binds to a cell surface marker of tau pathology, wherein the targeting ligand is conjugated to a liposome comprising an imaging agent, and imaging at least a portion of the subject to determine if that portion of the subject exhibits tau pathology. In some aspects, the portion of the subject includes a portion of the subject's brain. In some aspects, the imaging indicates a level of tau pathology sufficient to diagnose the subject as having early stage AD. In some aspects, the method further comprises providing prophylaxis or treatment of AD to the subject. In some aspects, the imaging agent is an MRI contrast enhancing agent and the level of binding is determined using MRI.

A method for detecting tau pathology is also provided, the method comprising: contacting a biological sample with an effective amount of a targeting ligand-liposome conjugate comprising a targeting ligand that specifically binds to a cell surface marker of tau pathology, wherein the targeting ligand is conjugated to a liposome comprising a detectable label; washing the biological sample to remove unbound targeting ligand liposome conjugate; and detecting tau pathology in the biological sample by determining the amount of detectable label remaining in the biological sample. In some aspects, the biological sample is a sample containing neural cells.

A targeting composition is also provided, the targeting composition comprising: a phospholipid linked to a polymer that is linked to a targeting ligand that specifically binds to a cell surface marker of tau pathology. In some aspects, the targeting ligand is an aptamer or stabilized aptamer. In some aspects, the targeting ligand is a thioaptamer. In some aspects, the aptamer or stabilized aptamer comprises a DNA nucleotide sequence selected from one or more of Tau_1 (SEQ ID NO: 5), Tau_3 (SEQ ID NO: 6), Tau_9 (SEQ ID NO: 7), Tau_11 (SEQ ID NO: 8), Tau_10 (SEQ ID NO: 9), Tau_13 (SEQ ID NO: 10), Tau_8 (SEQ ID NO: 11), Tau_4 (SEQ ID NO: 12), Tau_17 (SEQ ID NO: 13), Tau_5 (SEQ ID NO: 14), Tau_21 (SEQ ID NO: 15), Tau_25 (SEQ ID NO: 16), Tau_7 (SEQ ID NO: 17), Tau_31 (SEQ ID NO: 18), Tau_42 (SEQ ID NO: 19), Tau_14 (SEQ ID NO: 20), Tau_19 (SEQ ID NO: 21), Tau_15 (SEQ ID NO: 22), Tau_56 (SEQ ID NO: 23), Tau_34 (SEQ ID NO: 24), Tau_23 (SEQ ID NO: 25), Tau_99 (SEQ ID NO: 26), and Tau_102 (SEQ ID NO: 27).

An aptamer or stabilized aptamer is also provided, the aptamer or stabilized aptamer comprising a DNA nucleotide sequence selected from one or more of Tau_1 (SEQ ID NO: 5), Tau_3 (SEQ ID NO: 6), Tau_9 (SEQ ID NO: 7), Tau_11 (SEQ ID NO: 8), Tau_10 (SEQ ID NO: 9), Tau_13 (SEQ ID NO: 10), Tau_8 (SEQ ID NO: 11), Tau_4 (SEQ ID NO: 12), Tau_17 (SEQ ID NO: 13), Tau_5 (SEQ ID NO: 14), Tau_21 (SEQ ID NO: 15), Tau_25 (SEQ ID NO: 16), Tau_7 (SEQ ID NO: 17), Tau_31 (SEQ ID NO: 18), Tau_42 (SEQ ID NO: 19), Tau_14 (SEQ ID NO: 20), Tau_19 (SEQ ID NO: 21), Tau_15 (SEQ ID NO: 22), Tau_56 (SEQ ID NO: 23), Tau_34 (SEQ ID NO: 24), Tau_23 (SEQ ID NO: 25), Tau_99 (SEQ ID NO: 26), and Tau_102 (SEQ ID NO: 27).

### BRIEF DESCRIPTION OF THE FIGURES

The present invention may be more readily understood by reference to the following drawings wherein:

FIG. 1 provides an example depiction of an aptamer-functionalize d liposome.

FIG. 2 provides a schematic representation showing how the six alternate splicings of tau are sequentially phosphorylated and dephosphorylated by selected kinases and phosphatases. Imbalance between kinase and phosphatase activity, usually by phosphatase downregulation, results in an equilibrium shift to the right and formation of paired helical filaments and tau tangles.

FIGS. 3A-3H provide images showing preliminary Cell-SELEX on SH-SYSY cells to identify aptamers binding hyperphosphorylated cells. A: SH-SYSY cells treated with retinoic acid differentiate to a neuron-like phenotype with axonal and dendritic structures. B: After treatment with okadaic acid, cells avidly phosphorylate tau. Upper row: pTau Thr205/Ser202 stained by AT8 is nuclear. Lower row: pTau Ser396 stained by PHF-1 is predominantly cytosolic. C: Membrane bound aptamers at selected rounds of SELEX (1, 5, 10, 13, 17, 19, 21, 23, 26, and −ye control). Rounds 13 and 21 were negative selections against non-hyperphosphorylated cells, isolating the supernatant, thus ensuring specificity of binding. D: After IonTorrent sequencing, the 10 most abundant aptamers at round 26 and how they evolved through the screen. E: Dendrogram of the 20 most abundant sequences showing three distinct families. F: M-fold structures of Tau_1 (SEQ ID NO: 5), Tau_3 (SEQ ID NO: 6), and Tau_17 (SEQ ID NO: 13), showing similarity in structures. G, H: Tau_1 (SEQ ID NO: 5) and Tau_3 (SEQ ID NO: 6) aptamers with Cy5 labels bound to the membrane and axonal processes of hyperphosphorylated SH-SY5Y cells.

FIG. 4 provides a schematic representation showing the interactome of the MAPT gene and the protein targets of Tau_1 (SEQ ID NO: 5) and Tau_3 (SEQ ID NO: 6) aptamers: HSPD90, KRT6A, KRT6B, and VIM, from the VisANT database.

FIGS. 5A-5C, where A: provides an image showing thioaptamer Tau_3 (SEQ ID NO: 6) (Red) binds avidly to P301S mouse brain hippocampus tissue in regions where AT100 antibody also stains neurons (Green); B: demonstrates no correlation between AT8 staining (Green) and Tau_3 (SEQ ID NO: 6) binding (Red); and C: demonstrates that Tau_3 (SEQ ID NO: 6) binding is evident in normal mouse brain hippocampus.

FIG. 6 provides graphs and imaging showing examples of MRI using the GRE/45° FA sequence before and 4 days after intravenous injection of Tau_1 (SEQ ID NO: 5) aptamer targeted, Tau_3 (SEQ ID NO: 6) aptamer targeted, or untargeted control nanoparticle s, all bearing covalently conjugated Gd-DOTA as the contrast agent. Upper block: Brain axial pre and 4-day post images in 2-month old transgenic P301S mice. Lower block: Brain axial pre and 4-day post images in wild type siblings. (All images on same color map.) Also shown are the Receiver Operating Characteristic (ROC) curves and 95% confidence limits thereof for the Tau_1 (SEQ ID NO: 5) and Tau_3 (SEQ ID NO: 6) targeted particles calculated over the entire cohort of 20 animals tested for each treatment. Both Tau_1 (SEQ ID NO: 5) and Tau_3 (SEQ ID NO: 6) targeted particles show clear signal enhancement in the cortex, hippocampus, and hypothalamus areas in transgenic animals (yellow arrows), indicating that the particles are binding to neurons that are hyperphosphorylating tau. Both preparations show an accuracy of 80%, sensitivity ˜57%, and specificity ˜93%.

FIG. 7 provides an image showing a 2-month old P 301 S mouse treated with a Tau_3 (SEQ ID NO: 6) aptamer targeted nanoparticle. Axial spin-echo and 45° FA GRE images demonstrate thalamus/hypothalamus enhancement (red arrows), while T1 maps show significant hippocampal T1 shortening in addition (green arrow). The T1 mapping technique provides additional information and highlights quantifiable signal.

FIG. 8 provides images from 2-month old wild type (WT) and P301S transgenic (Tg) mice administered ADx-Tau_1 (SEQ ID NO: 5), ADx-Tau_3 (SEQ ID NO: 6), and untargeted ADx-Tau Control (ADx-Un) aptamer-bearing liposomal nanoparticles both before and four days post-contrast injection. Transgenic mice demonstrate high signal enhancement in cortical and hippocampal regions (gold arrows), particularly in the case of ADx-Tau_1 (SEQ ID NO: 5) aptamer injection. T1-weighted spin echo (T1w-SE) and fast spin echo inversion recovery (FSE-IR) sequences demonstrate in vivo efficacy.

FIG. 9A-9B provides post-mortem confirmation of hyperphosphorylated tau (pTau) in 7-month-old transgenic P301S mice brains via representative fluorescence microscopy images of DAPI nuclear staining and AF488 labeled AT100 binding to pTau in the cortical regions of (A) wild type and (B) transgenic mice. Magnification is 10×. AT100 images are shown on the same colorbar scale.

FIG. 10A-10D provides ROC curves generated on a six-point ordinal scale (empirical-green, fitted-blue), demonstrating higher accuracy for analysis of fast spin echo inversion recovery (FSE-IR) images relative to T1-weighted spin echo (Tlw-SE) images (42 animals tested, 7 animals per genotype and per formulation type). ROC curves are shown for the (A) ADx-Taul and (B) ADx-Tau3 formulations for the Tlw-SE sequence and the (C) ADx-Taul and (D) ADx-Tau3 formulations for the FSE-IR sequence.

To illustrate the invention, several embodiments of the invention will now be described in more detail. Reference will be made to the drawings, which are summarized above. Skilled artisans will recognize the embodiments provided herein have many useful alternatives that fall within the scope of the invention.

### DETAILED DESCRIPTION

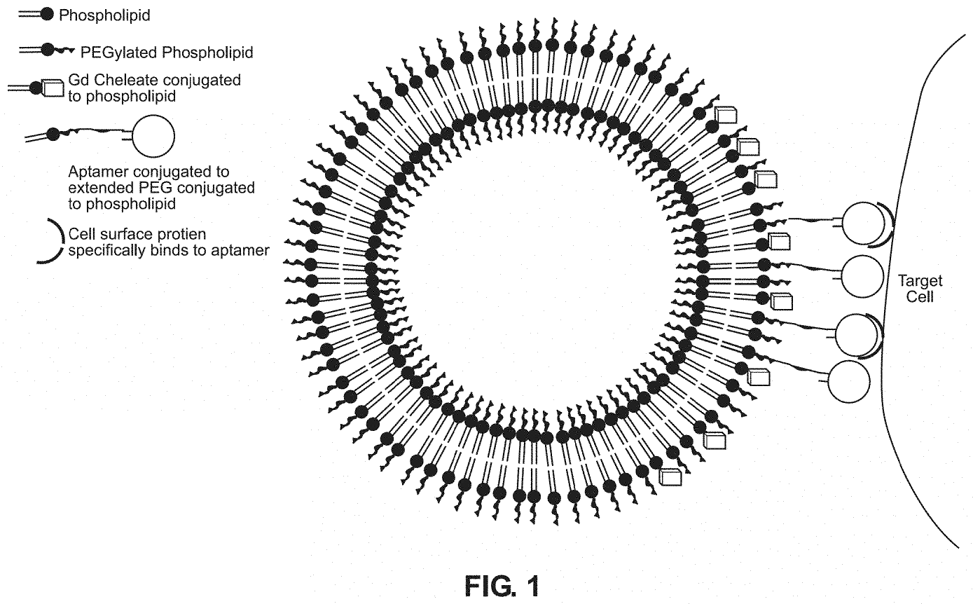

This disclosure provides methods and compositions for detecting tau pathology. The compositions for detecting tau pathology comprise a targeting ligand that specifically binds to a cell surface marker of tau pathology, wherein the targeting ligand is linked to a liposome that includes an imaging agent. See FIG. 1. The compositions may be used in a method for imaging tau pathology in a subject that comprises administering to the subject an effective amount of the composition to a subject and imaging at least a portion of the subject to determine if that portion of the subject exhibits tau pathology. The compositions may also be used to detect tau pathology in biological samples obtained from a subject.

### Definitions

Unless otherwise defined, all technical and scientific terms used herein have the same meaning as commonly understood by one of ordinary skill in the art to which this invention pertains. In case of conflict, the present specification, including definitions, will control.

Unless otherwise specified, “a,” “an,” “the,” “one or more of,” and “at least one” are used interchangeably. The singular forms “a”, “an,” and “the” are inclusive of their plural forms.

The recitations of numerical ranges by endpoints include all numbers subsumed within that range (e.g., 1 to 5 includes 1, 1.5, 2, 2.75, 3, 3.80, 4, 5, etc.).

The term “about,” when referring to a value or to an amount of mass, weight, time, volume, concentration, or percentage is meant to encompass variations of ±10% from the specified amount.

The terms “comprising” and “including” are intended to be equivalent and open-ended.

The phrase “consisting essentially of” means that the composition or method may include additional ingredients and/or steps, but only if the additional ingredients and/or steps do not materially alter the basic and novel characteristics of the claimed composition or method.

The phrase “selected from the group consisting of” is meant to include mixtures of the listed group.

An “effective” or a “detectably effective amount” of a composition means an amount sufficient to detect the presence of cell surface markers associated with tau pathology, or to yield an acceptable image using equipment that is available for clinical use. A detectably effective amount of a detecting or imaging agent may be administered in more than one injection. The detectably effective amount of the detecting or imaging agent may vary according to factors such as the degree of susceptibility of the individual, the age, sex, and weight of the individual, idiosyncratic responses of the individual, and the dosimetry. Detectably effective amounts of the detecting or imaging agent may also vary according to instrument and film-related factors. Optimization of such factors is well within the level of skill in the art. The amount of imaging agent used for diagnostic purposes and the duration of the imaging study will depend upon the specific imaging agent used, the body mass of the patient, the nature and severity of the condition being treated, the nature of therapeutic treatments under which the patient has gone, and on the idiosyncratic responses of the patient. Ultimately, the attending physician will decide the amount of imaging agent to administer to each individual patient and the duration of the imaging study.

The term “diagnosis” may encompass determining the nature of a disease in a subject, as well as determining the severity and probable outcome of the disease or episode of the disease, the prospect of recovery (prognosis), or both. “Diagnosis” may also encompass diagnosis in the context of rational therapy, in which the diagnosis guides therapy, including initial selection of therapy, modification of therapy (e.g., adjustment of dose and/or dosage regimen), and the like.

The term antigen refers to a molecule or a portion of a molecule capable of being bound by a targeting ligand. An antigen is typically also capable of inducing an animal to produce an antibody capable of binding to an epitope of that antigen. An antigen can have one or more than one epitope. The specific reaction referred to above is meant to indicate that the antigen will react, in a highly selective manner, with its corresponding antibody and not with the multitude of other antibodies that can be evoked by other antigens.

The term epitope refers to that portion of any molecule capable of being recognized by, and bound by, a targeting ligand such as an antibody or aptamer. In general, epitopes comprise chemically active surface groupings of molecules, for example, amino acids or sugar side chains, and have specific three-dimensional structural and specific charge characteristics.

The phrase “specifically binds” refers to a targeting ligand binding to a target structure, wherein the targeting ligand binds the target structure, or a sub-unit thereof, but does not bind to a biological molecule that is not the target structure, or the targeting ligand at least binds preferentially to the target structure. Targeting ligands (e.g., aptamers or antibodies) that specifically bind to a target structure, or a sub-unit thereof, may not cross-react with biological molecules that are outside of the target structure family. A targeting ligand specific for tau pathology can be a targeting ligand capable of binding to that specific protein with a specific affinity of between about 10⁻⁸ M and about 10⁻⁹ M. In some embodiments, an antibody or antibody fragment binds to a selected antigen with a specific affinity of greater than about 10⁻⁷ M, 10⁻⁸ M, 10⁻⁹ M, 10⁻¹⁰ M, or 10⁻¹¹ M, between about 10⁻⁸ M, 10⁻¹¹ M, 10⁻⁹ M, and 10⁻¹⁰ M, and between about 10⁻¹⁰ M-10⁻¹¹ M. In some aspects, specific activity is measured using a competitive binding assay as set forth in Ausubel FM, (1994). Current Protocols in Molecular Biology. Chichester: John Wiley and Sons (“Ausubel”), which is incorporated herein by reference.

The term “polynucleotide” refers to a nucleic acid sequence including DNA, RNA, and micro-RNA and can refer to markers that are either double-stranded or single-stranded. Polynucleotide can also refer to synthetic variants with alternative sugars such as locked nucleic acids.

### Imaging Tau Pathology

Imaging tau hyperphosphorylation is a novel approach to identify tau pathology. Previous attempts to image tau pathology have targeted the agglomerated protein itself. While it has been recognized that tau hyperphosphorylation is key to the formation of paired helical filaments and eventually, tau tangles, markers of hyperphosphorylation as a surrogate or precursor of tau pathology have not been investigated. The inventors have identified neuronal cells in a hyperphosphorylated state and their anatomical distribution in the brain, thus serving as a novel, sensitive, and specific marker of future tau pathology.

The proposed imaging agent will target a cell surface marker of tau hyperphosphorylation, eliminating the need for cell membrane permeability. Tau is an intracellular protein, and tau tangles are predominantly intracellular, with one exception: after neuronal death, tau tangles remain as “ghost tangles.” Thus, to date, all imaging markers of tau pathology had to, by necessity, penetrate the neuronal cell membrane and only then bind to their target. The only exception, of course, was to bind a ghost tangle. Thus, all tau imaging agents were restricted by membrane permeability. Binding ghost tangles would only serve to mark neuronal death, an advanced state of the disease. The claimed compositions and methods remove the requirement to penetrate the cell membrane and open the door to nanoparticle readouts that have high signal, but may have difficulty being internalized into the cell.

Identification of such cell surface markers could shed new light on the biology of tau fibrillation and tangle formation. The inventors conducted thioaptamer screens in “black-box” mode, with no knowledge of what the binding target was. They found that thioaptamers specifically binding to hyperphosphorylated cells bind KRT6A, KRT6B, HSP, and VIM.

Tau has numerous phosphorylation sites. For example, the longest isoform tau441 has 80 serine/threonine and five tyrosine sites that could be phosphorylated. Neurofibrillary tangles have been shown to contain tau phosphorylated in >40 sites. The phosphorylation of tau is mediated by several kinases, including GSK3β, CDK-5, CaMKII, PKA, and MARK p110. Known sites of tau phosphorylation include S199-202/T205, T231, T212/5214, and 5396, marked by the AT8, AT180, AT100, and PHF-1 antibodies. Phosphorylation is a sequential process, with each phosphorylation event at a specific site thought to prepare the molecule for the next event via exposure of a key binding pocket. The 5396/PHF-1 site is generally thought to be phosphorylated late in the process and is primarily associated with paired helical filaments and tangles, but recent observations suggest that the 5396/PHF-1 site may under certain conditions be phosphorylated earlier even than the 5199-202/T205 (AT8 stained) site.

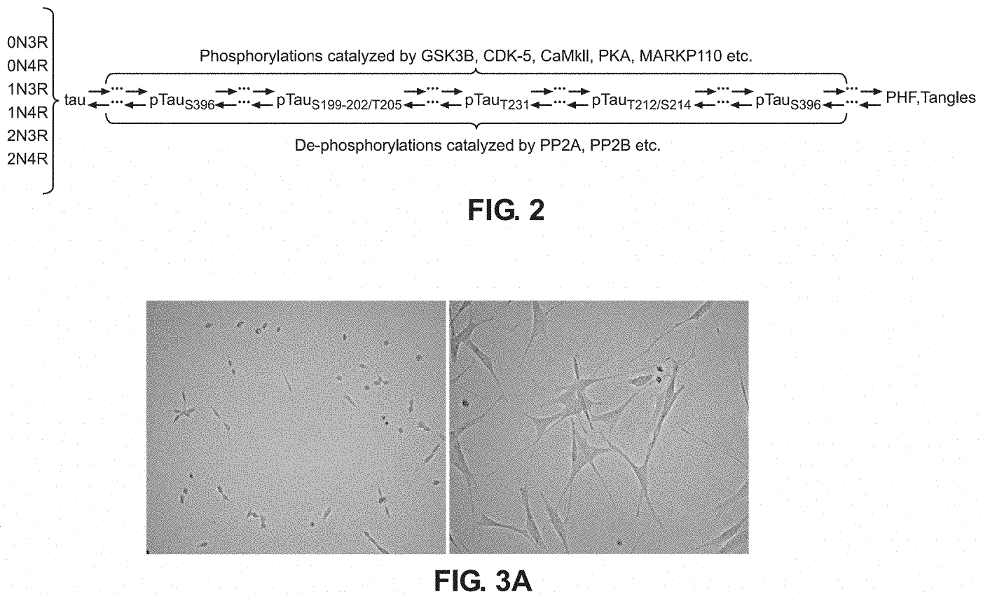

Tau dephosphorylation is mediated by protein phosphatases, of which PP2A accounts for over 70% of the function. The brains of AD patients have been shown to have less than 50% of normal PP2A activity, and this imbalance between kinase and phosphatase activity is suggested to be a significant contributor to tau hyperphosphorylation and the cascade to neurofibrillary tangle formation. See FIG. 2. Thus, PP2A was inhibited in a neuronal surrogate and thioaptamers conjugated to payload nanoparticles were used to identify surface markers of this hyperphosphorylated state.

### Compositions for Identifying Tau Pathology

In one aspect, a composition for identifying tau pathology is provided, the composition comprising a targeting ligand that specifically binds to a cell surface marker of tau pathology, wherein the targeting ligand is linked to a liposome comprising an imaging agent.

In some embodiments, the cell surface marker of tau pathology is a cell surface marker of tau hyperphosphorylation. Tau pathology refers to abnormal tau protein that results in taupathies. Tau pathology results from the hyperphosphorylation of tau protein. Normal tau contains 2-3 mol phosphate/mol protein, whereas hyperphosphorylated tau protein includes substantially higher levels of phosphate. Hyperphosphorylated tau leads to the formation of neurofibrillary tangles. Tau protein exists within the cell and is difficult to detect directly. However, the inventors have identified cell surface markers (i.e., epitopes) that are associated with the underlying tau pathology. In some embodiments, these cell surface markers are epitopes that have been identified using the Cell-SELEX method, in which neurons exhibiting tau pathology or cell models of neurons are used as targets for target ligands (e.g., aptamers). In some embodiments, the cell surface marker of tau pathology comprises a protein selected from KRT6A, KRT6B, HSP, and VIM.

### Targeting Ligands

The term “targeting ligand” as used herein includes any molecule that can be linked to the liposome for the purpose of engaging a specific target, and in particular for recognizing tau pathology. Examples of suitable targeting ligands include, but are not limited to, antibodies, antibody fragments, aptamers, and stabilized aptamers. In some embodiments, targeting ligands can be aptamers or stabilized aptamers that specifically bind to cell surface markers for tau pathology.

The targeting ligands of the invention are capable of specifically binding to cells exhibiting tau pathology. Specific binding refers to binding that discriminates between the selected target and other potential targets and binds with substantial affinity to the selected target. Substantial affinity represents a targeting ligand having a binding dissociation constant of at least about 10⁻⁸ mol/m³, but in other embodiments, the targeting ligand can have a binding dissociation constant of at least about 10⁻⁹ mol/m³, about 10⁻¹⁰ mol/m³, about 10⁻¹¹ mol/m³, or at least about 10⁻¹² mol/m³.

In some embodiments, the targeting ligand is an aptamer. An aptamer is a nucleic acid that binds with high specificity and affinity to a particular target molecule or cell structure, through interactions other than Watson-Crick base pairing. Suitable aptamers may be single stranded RNA, DNA, a modified nucleic acid, or a mixture thereof. The aptamers can also be in a linear or circular form. In some embodiments, the aptamers are single stranded DNA, while in other embodiments, they are single stranded RNA.

Aptamer functioning is unrelated to the nucleotide sequence itself, but rather is based on the secondary/tertiary structure formed by the polynucleotide, and aptamers are therefore best considered as non-coding sequences. Binding of a nucleic acid ligand to a target molecule is not determined by nucleic acid base pairing, but by the three-dimensional structure of the aptamer. In solution, the chain of nucleotides forms intramolecular interactions that fold the molecule into a complex three-dimensional shape. The shape of the nucleic acid ligand allows it to bind tightly against the surface of its target molecule. In addition to exhibiting remarkable specificity, nucleic acid ligands generally bind their targets with very high affinity, e.g., the majority of anti-protein nucleic acid ligands have equilibrium dissociation constants in the femtomolar to low nanomolar range.

The length of the aptamers suitable for use as targeting ligands is not particularly limited, and includes aptamers including about 10 to about 200 nucleotides, about 100 nucleotides or less, about 50 nucleotides or less, about 40 nucleotides or less, or about 35 nucleotides or less. In some embodiments, the aptamer has a size from about 15 to about 40 nucleotides. In addition, in almost all known cases, the various structural motifs that are involved in the non-Watson-Crick type of interactions involved in aptamer binding, such as hairpin loops, symmetric and asymmetric bulges, and pseudoknots, can be formed in nucleic acid sequences of 30 nucleotides or less.

In some aspects, the aptamers are stabilized aptamers that comprise a chemical modification to increase their stability. Modifications include, but are not limited to, those that provide other chemical groups that incorporate additional charge, polarizability, hydrophobicity, hydrogen bonding, electrostatic interaction, and fluxionality to the nucleic acid ligand bases or to the nucleic acid ligand as a whole. Such modifications include, but are not limited to, 2-position sugar modifications, 5-position pyrimidine modifications, 8-position purine modifications, modifications at exocyclic amines, substitution of 4-thiouridine, substitution of 5-bromo or 5-iodo-uracil, backbone modifications, phosphorothioate or alkyl phosphate modifications, methylations, unusual base-pairing combinations such as the isobases isocytidine and isoguanidine, and the like. Modifications can also include 3′ and 5′ modifications such as capping. In certain embodiments, the nucleic acid ligands comprise RNA molecules that are 2′-fluoro (2′-F) modified on the sugar moiety of pyrimidine residues.

Suitable stabilized aptamers can further include nucleotide analogs, such as, for example, xanthine or hypoxanthine, 5-bromouracil, 2-aminopurine, deoxyinosine, or methylated cytosine such as 5-methylcytosine, N4-methoxydeoxycytosine, and the like. Also included are bases of polynucleotide mimetics, such as methylated nucleic acids, e.g., 2′-O-methRNA, peptide nucleic acids, locked nucleic acids, modified peptide nucleic acids, and any other structural moiety that acts substantially like a nucleotide or base, for example, by exhibiting base-complementarity with one or more bases that occur in DNA or RNA.

In some embodiments, the stabilized aptamer comprises a thioaptamer. Thioaptamers are aptamers in which one or both of the non-bridging oxygen atoms have been substituted with sulfur. Oxygen-to-sulfur substitutions not only increases the stability of the aptamer, but in some cases also increases its binding affinity.

Typically, the targeting ligand (e.g., aptamer) is linked to a liposome comprising an imaging agent. However, a further aspect of the present invention is directed to the aptamers themselves. In some embodiments, the aptamer comprises a stabilized aptamer. In further embodiments, the stabilized aptamer is a thioaptamer. In some embodiments, the aptamer or stabilized aptamer specifically binds to tau pathology. Examples of suitable aptamers include those comprising a DNA nucleotide sequence selected from, and in some instances, selected from the group consisting of: Tau_1 (SEQ ID NO: 5), Tau_3 (SEQ ID NO: 6), Tau_9 (SEQ ID NO: 7), Tau_11 (SEQ ID NO: 8), Tau_10 (SEQ ID NO: 9), Tau_13 (SEQ ID NO: 10), Tau_8 (SEQ ID NO: 11), Tau_4 (SEQ ID NO: 12), Tau_17 (SEQ ID NO: 13), Tau_5 (SEQ ID NO: 14), Tau_21 (SEQ ID NO: 15), Tau_25 (SEQ ID NO: 16), Tau_7 (SEQ ID NO: 17), Tau_31 (SEQ ID NO: 18), Tau_42 (SEQ ID NO: 19), Tau_14 (SEQ ID NO: 20), Tau_19 (SEQ ID NO: 21), Tau_15 (SEQ ID NO: 22), Tau_56 (SEQ ID NO: 23), Tau_34 (SEQ ID NO: 24), Tau_23 (SEQ ID NO: 25), Tau_99 (SEQ ID NO: 26), and Tau_102 (SEQ ID NO: 27).

In some embodiments, the aptamers are positioned between two primer nucleotide sequences that facilitate amplification of the aptamer sequence, e.g., by Polymerase Chain Reaction (PCR). For example, in some embodiments, the DNA nucleotide sequence of the aptamer is positioned between the sequences GATATGTCTAGAGCCTCAGATCA (SEQ ID NO: 1) and CGGAGTTATGTTAGCAGTAGC (SEQ ID NO: 2). In other embodiments, the DNA nucleotide sequence of the aptamer is positioned between the sequences CGC TCG ATA GAT CGA GCT TCG (SEQ ID NO: 3) and GTC GAT CAC GCT CTA GAG CAC (SEQ ID NO: 4).

### Selection of Aptamers

In some embodiments, the aptamers or stabilized aptamers that specifically bind to a cell surface marker of tau pathology can be identified using the SELEX method. Suitable nucleic acid ligands can be identified using any methods known in the art, such as SELEX as described in Gold et al. (U.S. Pat. No. 5,270,163), the content of which is incorporated by reference herein in its entirety. Other nucleic acid ligand identification methods are shown in Gilman et al. (U.S. patent application number 2011/0104667), the content of which is incorporated by reference herein in its entirety. Identification of suitable aptamers is demonstrated in the Examples herein.

SELEX is a strategy developed for the identification of nucleic acids that can bind target molecules with high affinity and specificity through their three-dimensional conformation. The technique involves identification of rare nucleic acid molecules that have high affinity for a target molecule from a pool of random nucleic acids. The process is completed iteratively, with subsequent repeated rounds of selection and amplification. This procedure has proved to be extremely useful for the isolation of tight-binding oligonucleotide ligands (aptamers) for a number of target molecules, such as nucleic acid-binding proteins, non-nucleic acid-binding proteins, and certain small molecules. SELEX is an efficient screening method because iterative cycles of selection can be carried out using PCR.

The SELEX process generally involves defining a target molecule, such as a protein, a small molecule, or a supramolecular structure. A library of random oligonucleotides (˜1×10¹⁵ oligonucleotides) is created. The random pool of DNA generally has primer binding sites at the end of each oligonucleotide to provide an efficient way to find and PCR amplify oligonucleotides that bind to the target molecule. The target molecule is exposed to the oligonucleotide “library,” and a few of the oligonucleotides in the library will bind to the target, thus defining the target specific aptamers. The non-binding oligonucleotides are separated from the binding oligonucleotides.

Aptamer identification methods may involve single step separation of nucleic acids that bind the target molecule with the greatest affinity from nucleic acids that bind the target molecule with a lesser affinity and nucleic acids that do not bind the target molecule at all, thereby identifying the nucleic acid ligand of the target molecule. The selective separation protocols generate conditions in which the nucleic acids that bind the target molecule with a lesser affinity and nucleic acids that do not bind the target molecule at all cannot form complexes with the target molecule or can only form complexes with the target molecule for a short period of time. In contrast, the conditions of the separation protocols allow nucleic acids that bind the target molecule with greatest affinity to form complexes with the target molecule and/or bind the target molecule for the greatest period of time, thereby separating in a single step the nucleic acids with the greatest affinity for the target molecule, i.e., the nucleic acid ligands, from the remaining nucleic acids in the candidate mixture.

Separating can be accomplished by any of numerous methods that provide for selective single step separation of nucleic acids that bind the target molecule with greatest affinity from nucleic acids that bind the target molecule with a lesser affinity and nucleic acids that do not bind the target molecule. Suitable separating procedures include HPLC gradient elution and gel electrophoresis.

After incubation, the mixture is washed with buffer to remove unbound target molecules. The beads having bound target molecules are then incubated with the candidate mixture of nucleic acids. The beads having bound target molecules can be loaded into an HPLC column prior to incubating with the candidate mixture. If the beads having bound target molecules are loaded into the HPLC column prior to incubation with the candidate mixture, incubating of the candidate mixture and the target molecule occurs on the column.

After the candidate mixture has been incubated with the target molecules bound to the beads for sufficient time that bead/target molecule/nucleic acid complexes can form, an HPLC elution gradient is applied to the column in order to obtain the nucleic acid ligands of the target molecule. During the elution process, the effluent will be enriched in nucleic acid ligands of higher affinity for the target molecule, and eventually the final fractions contain the nucleic acid ligands of the highest affinity to the target molecule.

In some embodiments, the aptamers or stabilized aptamers that specifically bind to a cell surface marker of tau pathology can be identified using a Cell-SELEX method. Cell-SELEX elects aptamers by use of complex whole cells as targets. A counterselection strategy is then used to isolate aptamer sequences that interact only with the target cell and not with the control cells. Through this process, a group of cell-specific aptamers can be selected in a relatively short period, even if it is not known which target molecules are present on the cell surface and which membrane molecules might play an important role in the pathology being detected.

In some embodiments, the aptamers or stabilized aptamers that specifically bind to a cell surface marker of tau pathology can be identified using a Conjugate-SELEX method. Conjugate-SELEX is a modification of the basic SELEX procedure in which the entire aptamer-liposome conjugate is evaluated for affinity, rather than evaluating the aptamers in isolation.

### Sequencing

After identification, the aptamers may be sequenced. Sequencing may be by any method known in the art. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, the 454 sequencing method, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Sequencing may be by any method known in the art. See for example Sanger et al. (Proc Natl Acad Sci USA, 74(12): 5463 67, 1977), Maxam et al. (Proc. Natl. Acad. Sci., 74: 560-564, 1977), and Drmanac, et al. (Nature Biotech., 16:54-58, 1998), which references describe example conventional ensemble sequencing techniques. Also see Lapidus et al. (U.S. Pat. No. 7,169,560), Quake et al. (U.S. Pat. No. 6,818,395), Harris (U.S. Pat. No. 7,282,337), Quake et al. (U.S. patent application number 2002/0164629), and Braslaysky, et al., (PNAS (USA), 100: 3960-3964, 2003), which references describe example single molecule sequencing by synthesis techniques. The contents of each of these references is incorporated by reference herein in its entirety.

The inventors have identified aptamers that specifically bind to tau pathology. Examples of these aptamers are described in Table 1. Accordingly, in some embodiments, the aptamer or stabilized aptamer comprises a DNA nucleotide sequence selected from, including selected from the group consisting of: Tau_1 (SEQ ID NO: 5), Tau_3 (SEQ ID NO: 6), Tau_9 (SEQ ID NO: 7), Tau_11 (SEQ ID NO: 8), Tau_10 (SEQ ID NO: 9), Tau_13 (SEQ ID NO: 10), Tau_8 (SEQ ID NO: 11), Tau_4 (SEQ ID NO: 12), Tau_17 (SEQ ID NO: 13), Tau_5 (SEQ ID NO: 14), Tau_21 (SEQ ID NO: 15), Tau_25 (SEQ ID NO: 16), Tau_7 (SEQ ID NO: 17), Tau_31 (SEQ ID NO: 18), Tau_42 (SEQ ID NO: 19), Tau_14 (SEQ ID NO: 20), Tau_19 (SEQ ID NO: 21), Tau_15 (SEQ ID NO: 22), Tau_56 (SEQ ID NO: 23), Tau_34 (SEQ ID NO: 24), Tau_23 (SEQ ID NO: 25), Tau_99 (SEQ ID NO: 26), and Tau_102 (SEQ ID NO: 27). In a further embodiment, the aptamer or stabilized aptamer comprises the DNA nucleotide sequence Tau_1 (SEQ ID NO: 5), Tau_3 (SEQ ID NO: 6), or both.

TABLE 1
Aptamers that Specifically Bind to Tau Pathology

| | | SEQ |
|---|---|---|
| Identifier | Nucleotide Sequence | ID NO |
| Tau_1 | CCCCCCACGGTCTCCGCTCCACAAGTTCAC | SEQ ID |
| | | NO: 5 |
| Tau_3 | CCCCCCACGGTCTCCGCTCCACAAGTCCAC | SEQ ID |
| | | NO: 6 |
| Tau_9 | CCCCCCACGGTCTCCGCTCCACAGGTTCAC | SEQ ID |
| | | NO: 7 |
| Tau_11 | CCCCCCCACGGTCTCCGCTCCACAAGTTCA | SEQ ID |
| | | NO: 8 |
| Tau_10 | CTCGTGGGTGTGTGGTGGTGTTGTTGTGTG | SEQ ID |
| | | NO: 9 |
| Tau_13 | CCCCCCACGGTCTCCGCTCCACAAGCCCAC | SEQ ID |
| | | NO: 10 |
| Tau_8 | CTCGTCCCACCACAACATCATCTCAACGCC | SEQ ID |
| | | NO: 11 |
| Tau_4 | CTCGTCCCACCACAACATTATCTCAACGCC | SEQ ID |
| | | NO: 12 |
| Tau_17 | CTCGTGGGTGTACGGTGGTGTTGTTGTGTG | SEQ ID |
| | | NO: 13 |
| Tau_5 | CTCCGACGGGATGTTCGATGAGCACACACT | SEQ ID |
| | | NO: 14 |
| Tau_21 | CCCCCCCACGGTCTCCGCTCCACAAGTCCA | SEQ ID |
| | | NO: 15 |
| Tau_25 | CCCCCCACGGTCTCCGCTCCACAGGTCCAC | SEQ ID |
| | | NO: 16 |
| Tau_7 | CCCCCATTGGCTCCGCTCCACACAGCTTCA | SEQ ID |
| | | NO: 17 |
| Tau_31 | CCCCCCACGGTCTCCGCTCCACAAGCTCAC | SEQ ID |
| | | NO: 18 |
| Tau_42 | CCCCCCCACGGTCTCCGCTCCACAGGTTCA | SEQ ID |
| | | NO: 19 |
| Tau_14 | CTCGTCCCACCACAACATTGTCTCAACGCC | SEQ ID |
| | | NO: 20 |
| Tau_19 | CTCGTCCCACCACAACACCATCTCAACGCC | SEQ ID |
| | | NO: 21 |
| Tau_15 | CTCCGACGGGGTGTTCGATGAGCACACACT | SEQ ID |
| | | NO: 22 |
| Tau_56 | CCCCCCGCGGTCTCCGCTCCACAAGTTCAC | SEQ ID |
| | | NO: 23 |
| Tau_34 | TGGGTGTGTGGTGGTGTTGTTGTGTGGGTG | SEQ ID |
| | | NO: 24 |
| Tau_23 | CTCGCCCCACCACAACATCATCTCAACGCC | SEQ ID |
| | | NO: 25 |
| Tau_99 | CCCCCCACGGTCTCCGCTCCACAAGTTCGC | SEQ ID |
| | | NO: 26 |
| Tau_102 | CCCCCCCACGGTCTCCGCTCCACAAGCTCA | SEQ ID |
| | | NO: 27 |

In some embodiments, the targeting ligand is an antibody that specifically binds to tau pathology. The term “antibody” as used herein refers to a protein of the kind that is produced by activated B cells after stimulation by an antigen and can bind specifically to the antigen, thereby promoting an immune response in biological systems. Full antibodies typically comprise four subunits including two heavy chains and two light chains. The term antibody includes natural and synthetic antibodies, including but not limited to, monoclonal antibodies, polyclonal antibodies, or fragments thereof. Suitable antibodies include IgA, IgD, IgG1, IgG2, IgG3, IgM, and the like. Suitable fragments include Fab Fv, Fab′ F(ab′)₂, and the like. A monoclonal antibody is an antibody that specifically binds to, and is thereby defined as, complementary to a single particular spatial and polar organization of an epitope. In some forms, monoclonal antibodies can also have the same structure. A polyclonal antibody refers to a mixture of different monoclonal antibodies. In some forms, polyclonal antibodies can be a mixture of monoclonal antibodies where at least two of the monoclonal antibodies bind to a different antigenic epitope. The different antigenic epitopes can be on the same target, different targets, or a combination thereof. Antibodies can be prepared by techniques that are well known in the art, such as immunization of a host and collection of sera (polyclonal) or by preparing continuous hybridoma cell lines and collecting the secreted protein (monoclonal).

### Targeting Ligand Conjugates

In some embodiments, the targeting ligands (e.g., aptamers) are linked to a liposome or other vehicles for targeted delivery of an imaging or detecting agent. For example, imaging or detecting agents can be encapsulated within the liposome. Employing such techniques, the tau pathology-specific aptamers of the invention conjugated to a liposomal vesicle can provide targeted delivery of imaging or detecting agents to cells expressing tau pathology. In some embodiments, a single targeting ligand is linked to a liposome. In other embodiments, a plurality of targeting ligands are linked to the liposome (e.g., Tau_1 (SEQ ID NO: 5) and Tau-3).

The term “liposome” as used herein indicates a vesicular structure comprised of lipids. The lipids typically have a tail group comprising a long hydrocarbon chain and a hydrophilic head group. The lipids are arranged to form a lipid bilayer (i.e., membrane) with an inner aqueous environment suitable to contain an agent (e.g., imaging agent) to be delivered. Such liposomes present an outer surface that may comprise suitable targeting ligands that specifically bind to cell surface markers of tau pathology. A suitable liposome platform may be, for example, the “ADx” platform from Alzeca Biosciences, comprising hydrogenated soy L-α-phosphatidylcholine (HSP C), cholesterol (Chol), 1,2-distearoyl-sn-glycero-3-phosphoethanolamine-N-(methoxy (polyethylene glycol)-2000) (DSPE-mPEG2000), and Gd(III)-DSPE-DOTA (the macrocyclic gadolinium imaging moiety, Gd(III)-DOTA, conjugated to a phospholipid, 1,2-Distearoyl-sn-glycero-3-phosphorylethanolamine, DSPE), as well as the entity used to conjugate the targeting ligand, DSPE-PEG-3400 (1,2-distearoyl-sn-glycero-3-phosphoethanolamine-N-[maleimide(polyethylene glycol)-3400]).

In some embodiments, the membrane of the liposome may comprise at least three types of phospholipids. The membrane may comprise a first phospholipid, which may be unmodified. Suitable first phospholipids include those disclosed in U.S. Pat. Nos. 7,785,568 and 10,537,649, each of which is incorporated by reference herein in its entirety. In one embodiment, the first phospholipid is HSPC. The membrane may include a second phospholipid that may be derivatized with a first polymer. Suitable polymer-derivatized second phospholipids include those disclosed in U.S. Pat. Nos. 7,785,568 and 10,537,649. In one embodiment, the second phospholipid that is derivatized with a first polymer is DSPE-mPEG2000. The membrane may include a third phospholipid that is derivatized with a second polymer, the second polymer ultimately being conjugated to the targeting ligand. Suitable polymer-derivatized third phospholipids include those disclosed in U.S. Pat. Nos. 7,785,568 and 10,537,649. One embodiment, the third phospholipid that is derivatized with a second polymer is DSPE-PEG-3400.

In some embodiments, the membrane may comprise a sterically bulky excipient that is capable of stabilising the liposome. Suitable excipients include those disclosed in U.S. Pat. Nos. 7,785,568 and 10,537,649. In one embodiment, the sterically bulky excipient that is capable of stabilizing the liposome is cholesterol.

In some embodiments, the phospholipid moiety in the phospholipid-polymer-targeting ligand conjugate may be represented by the following structural formula:

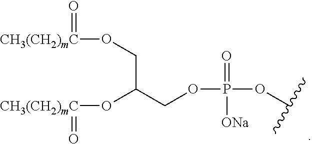

The variable m may be one of: 12, 13, 14, 15, 16, 17, or 18. For example, m may be 14 or 16. In various embodiments, the phospholipid moiety in any of the first phospholipid, the second phospholipid, and the phospholipid-polymer-targeting ligand conjugate may be one of: HSPC, DPPC, DSPE, DSPC, or DPPE.

In some embodiments, the polymer moiety in the phospholipid-polymer-targeting ligand conjugate is a polyol. Structural units forming polymers containing polyols comprise monomeric polyols such as pentaerythritol, ethylene glycol, and glycerin. Example polymers containing polyols comprise polyesters, polyethers, and polysaccharides. Example suitable polyethers include, but are not limited to, diols, such as diols with the general formula HO—(CH₂CH₂O)ₚ—H with p≥1, for example, polyethylene glycol, polypropylene glycol, and poly(tetramethylene ether) glycol. Suitable polysaccharides include, but are not limited to, cyclodextrins, starch, glycogen, cellulose, chitin, and 13-Glucans. Suitable polyesters include, but are not limited to, polycarbonate, polybutyrate, and polyethylene terephthalate, all terminated with hydroxyl end groups. Example polymers containing polyols comprise polymers of about 500,000 Da or less molecular weight, including from about 300 to about 100,000 Da.

In some embodiments, the polymer moiety in the phospholipid-polymer-targeting ligand conjugate comprises a hydrophilic poly(alkylene oxide) polymer. The hydrophilic poly(alkylene oxide) may include between about 10 and about 100 repeat units, and having, e.g., a molecular weight ranging from about 500-10,000 Da. The hydrophilic poly(alkylene oxide) may comprise, for example, poly(ethylene oxide), poly (propylene oxide), and the like. The polymer moiety in the phospholipid-polymer-targeting ligand conjugate may be conjugated to the phospholipid moiety via an amide or carbamate group. The polymer moiety in the phospholipid-polymer-targeting ligand conjugate may be conjugated via an amide, carbamate, poly (alkylene oxide), triazole, combinations thereof, and the like. For example, the polymer moiety in the phospholipid-polymer-targeting ligand conjugate may be represented by one of the following structural formulas:

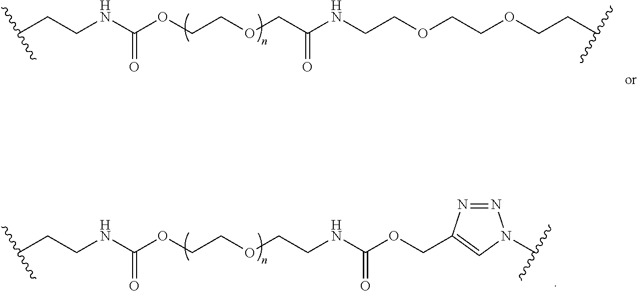

The variable n may be any integer from about 10 to about 100, for example, about 60 to about 100, about 70 to about 90, about 75 to about 85, or about 77.

In some embodiments, the phospholipid-polymer moiety in the phospholipid-polymer-targeting ligand conjugate may be represented by one of the following structural formulas:

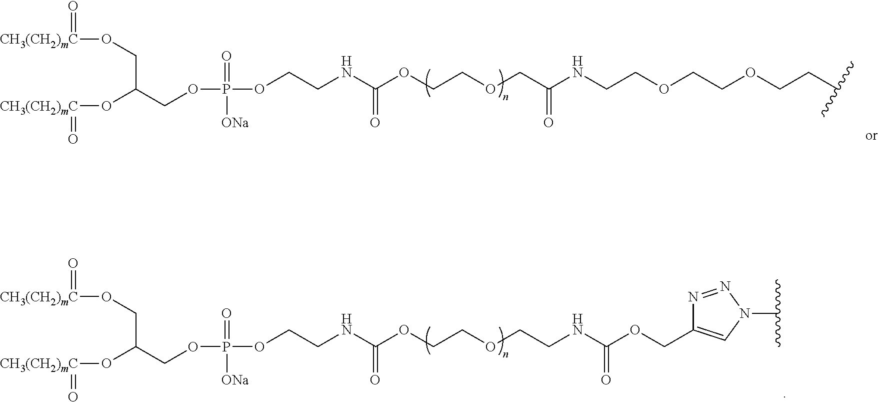

The variable n may be any integer from about 10 to about 100, for example, about 60 to about 100, about 70 to about 90, about 75 to about 85, or about 77. The variable m may be one of: 12, 13, 14, 15, 16, 17, or 18. For example, n may be 77 and m may be 14. In another example, n may be 77 and m may be 16.

The targeting ligands (e.g., aptamers) may be connected to one or more polymer (e.g. PEG) moieties of the phospholipid-polymer-targeting ligand conjugate, with or without one or more linkers. The PEG moieties may be any type of PEG moiety (linear, branched, multiple branched, star shaped, comb shaped, or a dendrimer) and have any molecular weight. The same or different linkers or no linkers may be used to connect the same or different PEG moieties to an aptamer. Commonly known linkers include, but are not limited to, amines, thiols, and azides, and can include a phosphate group. For example, in some embodiments, the targeting ligand is linked to polyethylene glycol that is conjugated to a phospholipid that associates with the liposome.

In some embodiments, the liposomes include a membrane, the membrane comprising: a first phospholipid selected from HSPC, DPPC, DSPE, DSPC, and DPPE; cholesterol; DPPC, DSPE, DSPC, and/or DPPE derivatized with PEG; DPPC, DSPE, DSPC, and/or DPPE derivatized with PEG and a targeting ligand that specifically binds to a cell surface marker of tau pathology; and an imaging agent that is encapsulated by or bound to the membrane. In further embodiments, the targeting ligand is a thioaptamer, and the imaging agent is an MRI contrast enhancing agent.

In some aspects, the present invention provides a targeting composition. The targeting composition includes a phospholipid linked to a polymer that is linked to a targeting ligand that specifically binds to a cell surface marker of tau pathology. The phospholipid can be any of the phospholipids described herein. In some embodiments, the phospholipid comprises one or more of DPPC, DSPE, DSPC, and DPPE. Likewise, the polymer can be any of the polymers (e.g., polyols) described herein. In some embodiments, the polymer is polyethylene glycol.

### Imaging or Detecting Agents

The composition for detecting tau pathology described herein may include an imaging or detecting agent. The imaging or detecting agent is generally associated with the liposome portion of the composition. The imaging or detecting agent can be held within the liposome, or it can be conjugated to the liposome. In one embodiment, the imaging or detecting agent is linked to a polymer that is linked to a phospholipid that associates with the membrane forming the liposome. In one embodiment, the imaging or detecting agent is linked to a polymer that is linked to a phospholipid that associates with the membrane forming the liposome comprises Gd(III)-DSPE-DOTA.

In some embodiments, the composition for detecting tau pathology includes a detecting agent. Examples of detecting agents include GFP, biotin, cholesterol, dyes such as fluorescence dyes, electrochemically active reporter molecules, and compositions comprising radioactive residues, such as radionuclides suitable for PET (positron emission tomography) detection, e.g., ¹⁸F, ¹¹C, ¹³N, ¹⁵O, ⁸²Rb or ⁶⁸Ga.

In some embodiments, the composition for detecting tau pathology comprises an imaging agent. Imaging agents differ from detecting agents in that they not only indicate the presence of tau pathology, but are suitable for use with imaging methods that allow an image of a region of tissue exhibiting the tau pathology to be created and displayed. Examples of imaging agents include near infrared imaging agents, positron emission tomography imaging agents, single-photon emission tomography agents, fluorescent compositions, radioactive isotopes, and MRI contrast agents.

In some embodiments, the imaging agent is an MRI contrast enhancing agent. Disease detection using MRI is often difficult because areas of disease have similar signal intensity compared to surrounding healthy tissue. In the case of MRI, the imaging agent can also be referred to as a contrast agent. The MRI contrast enhancing agent may be a nonradioactive MRI contrast enhancing agent that may be at least one of encapsulated by or bound to the membrane. For example, the nonradioactive MRI contrast enhancing agent may be both encapsulated by and bound to the membrane, e.g., to provide a dual contrast agent liposome. The liposomal composition may be characterized by a per-particle relaxivity in mM⁻¹s⁻¹ of at least about one or more of about: 100,000, 125,000, 150,000, 165,000, 180,000, 190,000, and 200,000. Detecting the liposomal formulation may include detecting using MRI in a magnetic field range of, for example, between about 1 T to about 3.5 T, or about 1.5 to about 3T. The nonradioactive MRI contrast enhancing agent may include gadolinium. Suitable nonradioactive MRI contrast enhancing agent may include Gd(III)-DSPE-DOTA and (diethylenetriaminepentaacetic acid)-bis(stearylamide), gadolinium salt (Gd-DTPA-BSA). Gadolinium paramagnetic chelates such as GdDTPA, GdDOTA, GdHPDO3A, GdDTPA-BMA, and GdDTPA-BSA are also suitable known MRI contrast agents. See U.S. Pat. No. 5,676,928 issued to Klaveness et al., which is incorporated by reference herein in its entirety.

### Methods of Imaging or Detecting Tau Pathology

In another aspect, the present invention provides a method for imaging tau pathology in a subject. The method comprises administering to the subject a detectably effective amount of a targeting ligand-liposome conjugate comprising a targeting ligand that specifically binds to a cell surface marker of tau pathology, wherein the targeting ligand is conjugated to a liposome comprising an imaging agent, and imaging at least a portion of the subject to determine if that portion of the subject exhibits tau pathology.

In some aspects, a method for imaging tau pathology in a subject is provided, the method comprising:

(i) administering to the subject a detectably effective amount of a targeting ligand-liposome conjugate comprising a targeting ligand that specifically binds to a cell surface marker of tau pathology, wherein the targeting ligand is conjugated to a liposome comprising an imaging agent; and

(ii) imaging at least a portion of the subject to determine if the portion exhibits tau pathology. The targeting ligand may comprise an aptamer. The targeting ligand may comprise a stabilized aptamer. The targeting ligand may comprise a thioaptamer. The targeting ligand may comprise a DNA nucleotide sequence selected from the group consisting of Tau_1 (SEQ ID NO: 5), Tau_3 (SEQ ID NO: 6), Tau_9 (SEQ ID NO: 7), Tau_11 (SEQ ID NO: 8), Tau_10 (SEQ ID NO: 9), Tau_13 (SEQ ID NO: 10), Tau_8 (SEQ ID NO: 11), Tau_4 (SEQ ID NO: 12), Tau_17 (SEQ ID NO: 13), Tau_5 (SEQ ID NO: 14), Tau_21 (SEQ ID NO: 15), Tau_25 (SEQ ID NO: 16), Tau_7 (SEQ ID NO: 17), Tau_31 (SEQ ID NO: 18), Tau_42 (SEQ ID NO: 19), Tau_14 (SEQ ID NO: 20), Tau_19 (SEQ ID NO: 21), Tau_15 (SEQ ID NO: 22), Tau_56 (SEQ ID NO: 23), Tau_34 (SEQ ID NO: 24), Tau_23 (SEQ ID NO: 25), Tau_99 (SEQ ID NO: 26), and Tau_102 (SEQ ID NO: 27). The portion may include a portion of the subject's brain. The imaging may indicate a level of tau pathology sufficient to diagnose the subject as having early stage Alzheimer's disease. The imaging agent may be an MRI contrast enhancing agent, and the level of binding may be determined using MRI. The cell surface marker of tau pathology may comprise a protein selected from KRT6A, KRT6B, HSP, and VIM. The liposome may comprise a membrane, the membrane comprising:

a first phospholipid;

a sterically bulky excipient that is capable of stabilizing the liposome;

a second phospholipid that is derivatized with a first polymer;

a third phospholipid that is derivatized with a second polymer, the second polymer being conjugated to the targeting ligand; and

the imaging agent, which is encapsulated by or bound to the membrane.

In some aspects, a method for detecting tau pathology is provided, the method comprising:

contacting a biological sample with an effective amount of a targeting ligand-liposome conjugate comprising a targeting ligand that specifically binds to a cell surface marker of tau pathology, wherein the targeting ligand is conjugated to a liposome comprising a detectable label;

washing the biological sample to remove unbound targeting ligand liposome conjugate; and

detecting tau pathology in the biological sample by determining the amount of detectable label remaining in the biological sample. The biological sample may comprise neural cells. The targeting ligand may comprise an aptamer. The targeting ligand may comprise a stabilized aptamer. The targeting ligand may comprise a thioaptamer. The targeting ligand may comprise a DNA nucleotide sequence selected from the group consisting of Tau_1 (SEQ ID NO: 5), Tau_3 (SEQ ID NO: 6), Tau_9 (SEQ ID NO: 7), Tau_11 (SEQ ID NO: 8), Tau_10 (SEQ ID NO: 9), Tau_13 (SEQ ID NO: 10), Tau_8 (SEQ ID NO: 11), Tau_4 (SEQ ID NO: 12), Tau_17 (SEQ ID NO: 13), Tau_5 (SEQ ID NO: 14), Tau_21 (SEQ ID NO: 15), Tau_25 (SEQ ID NO: 16), Tau_7 (SEQ ID NO: 17), Tau_31 (SEQ ID NO: 18), Tau_42 (SEQ ID NO: 19), Tau_14 (SEQ ID NO: 20), Tau_19 (SEQ ID NO: 21), Tau_15 (SEQ ID NO: 22), Tau_56 (SEQ ID NO: 23), Tau_34 (SEQ ID NO: 24), Tau_23 (SEQ ID NO: 25), Tau_99 (SEQ ID NO: 26), and Tau_102 (SEQ ID NO: 27). The method may further comprise the step of obtaining the biological sample from a subject.

The term “subject” refers to an animal such as a vertebrate or invertebrate animal. In some embodiments, the subject is a mammal, including, but not limited to, primates, including simians and humans, equines (e.g., horses), canines (e.g., dogs), felines, various domesticated livestock (e.g., ungulates, such as swine, pigs, goats, sheep, and the like), as well as domesticated pets and animals maintained in zoos. In some embodiments, the subject is a human subject. In some embodiments, the subject is a subject having an increased risk of developing AD. Risk factors for Alzheimer's disease include genetic predisposition, smoking, diabetes, a history of head injuries, depression, and hypertension. See Burns A, Iliffe S., BMJ., 338: b158 (2009)

The targeting ligand-liposome conjugate can include any of the features described herein. For example, in some embodiments, the targeting ligand is an aptamer or stabilized aptamer, while in further embodiments, the targeting ligand is a thioaptamer. In yet further embodiments, the aptamer or stabilized aptamer used in the method comprises a DNA nucleotide sequence selected from, including selected from the group consisting of Tau_1 (SEQ ID NO: 5), Tau_3 (SEQ ID NO: 6), Tau_9 (SEQ ID NO: 7), Tau_11 (SEQ ID NO: 8), Tau_10 (SEQ ID NO: 9), Tau_13 (SEQ ID NO: 10), Tau_8 (SEQ ID NO: 11), Tau_4 (SEQ ID NO: 12), Tau_17 (SEQ ID NO: 13), Tau_5 (SEQ ID NO: 14), Tau_21 (SEQ ID NO: 15), Tau_25 (SEQ ID NO: 16), Tau_7 (SEQ ID NO: 17), Tau_31 (SEQ ID NO: 18), Tau_42 (SEQ ID NO: 19), Tau_14 (SEQ ID NO: 20), Tau_19 (SEQ ID NO: 21), Tau_15 (SEQ ID NO: 22), Tau_56 (SEQ ID NO: 23), Tau_34 (SEQ ID NO: 24), Tau_23 (SEQ ID NO: 25), Tau_99 (SEQ ID NO: 26), and Tau_102 (SEQ ID NO: 27).

In some embodiments, the present invention may provide a method for generating an image of a tissue region of a subject, by administering to the subject a detectably effective amount of the composition for detecting tau pathology, and generating an image of a portion of the subject (i.e., a tissue region) to which the composition including the imaging agent has distributed. To generate an image of the tissue region, it is necessary for a detectably effective amount of imaging agent to reach the tissue region of interest, but it is not necessary that the imaging agent be localized in this region alone. However, in some embodiments, the compositions including the imaging agents are targeted or administered locally such that they are present primarily in the tissue region of interest. Examples of images include two-dimensional cross-sectional views and three-dimensional images. In some embodiments, a computer is used to analyze the data generated by the imaging agents in order to generate a visual image. The tissue region or portion of the subject can be an organ of a subject such as the brain heart, lungs, or blood vessels. In other embodiments, the portion of the subject can be a tissue region known to include neural cells, such as the brain. Examples of imaging methods include optical imaging, fluorescence imaging, computed tomography, positron emission tomography, single photon emission computed tomography, and MRI. Any other suitable type of imaging methodology known by those skilled in the art is contemplated.

In some embodiments, the imaging agent is an MRI contrast enhancing agent, and the level of binding is determined using MRI. MRI is a medical application of nuclear magnetic resonance, and forms pictures of the anatomy and physiological processes of the body using strong magnetic fields, magnetic field gradients, and radio waves to generate images of a portion of a subject. MRI is commonly used for neuroimaging, cardiovascular imaging, musculoskeletal imaging, liver imaging, and gastrointestinal imaging. MRI for imaging of anatomical structures or blood flow does not require contrast agents as the varying properties of the tissues or blood provide natural contrasts. However, for more specific types of imaging, exogenous contrast agents may be administered. For a review of neural imaging techniques, see Mehrabian et al. (Front Oncol., 9:440 (2019).

Another aspect of the invention may provide a method for detecting tau pathology. The method includes contacting a biological sample with an effective amount of a targeting ligand-liposome conjugate comprising a targeting ligand that specifically binds to a cell surface marker of tau pathology, wherein the targeting ligand is conjugated to a liposome comprising a detectable label, washing the biological sample to remove unbound targeting ligand liposome conjugate, and detecting tau pathology in the biological sample by determining the amount of detectable label remaining in the biological sample.

The targeting ligand-liposome conjugate can include any of the features described herein. For example, in some embodiments, the targeting ligand is an aptamer or stabilized aptamer, while in further embodiments, the targeting ligand is a thioaptamer. In yet further embodiments, the aptamer or stabilized aptamer used in the method comprises a DNA nucleotide sequence selected from, including selected from the group consisting of, Tau_1 (SEQ ID NO: 5), Tau_3 (SEQ ID NO: 6), Tau_9 (SEQ ID NO: 7), Tau_11 (SEQ ID NO: 8), Tau_10 (SEQ ID NO: 9), Tau_13 (SEQ ID NO: 10), Tau_8 (SEQ ID NO: 11), Tau_4 (SEQ ID NO: 12), Tau_17 (SEQ ID NO: 13), Tau_5 (SEQ ID NO: 14), Tau_21 (SEQ ID NO: 15), Tau_25 (SEQ ID NO: 16), Tau_7 (SEQ ID NO: 17), Tau_31 (SEQ ID NO: 18), Tau_42 (SEQ ID NO: 19), Tau_14 (SEQ ID NO: 20), Tau_19 (SEQ ID NO: 21), Tau_15 (SEQ ID NO: 22), Tau_56 (SEQ ID NO: 23), Tau_34 (SEQ ID NO: 24), Tau_23 (SEQ ID NO: 25), Tau_99 (SEQ ID NO: 26), and Tau_102 (SEQ ID NO: 27).

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers, and the like. Similarly, enzymatic labels may be detected by providing the appropriate substrates for the enzyme and detecting the resulting reaction product. The level of detected label can be compared to control levels to determine if the biological sample exhibits an increased level of cell surface markers for tau pathology.

Biological samples can be mammalian body fluids, sera such as blood (including whole blood, as well as its plasma and serum), CSF (spinal fluid), urine, sweat, saliva, tears, pulmonary secretions, breast aspirate, prostate fluid, seminal fluid, stool, cervical scraping, cysts, amniotic fluid, intraocular fluid, mucous, moisture in breath, animal tissue, cell lysates, tumor tissue, hair, skin, buccal scrapings, nails, bone marrow, cartilage, prions, bone powder, ear wax, etc., or even from external or archived sources such as tumor samples (i.e., fresh, frozen, or paraffin-embedded). Samples, such as body fluids or sera, obtained during the course of clinical trials may be suitable. In some embodiments, the biological sample comprises CSF or a sample containing neural cells, such as a neural (e.g., brain) tissue sample.

A biological sample may be fresh or stored. Samples can be stored for varying amounts of time, such as being stored for an hour, a day, a week, a month, or more than a month. The biological sample may be expressly obtained for use in the methods of the invention or may be a sample obtained for another purpose which can be subsampled for the assays of this invention. In some embodiments, it may be useful to filter, centrifuge, or otherwise pre-treat the biological sample to remove impurities or other undesirable matter that could interfere with analysis of the biological sample.

In some embodiments, the method includes the step of obtaining the biological sample from a subject. The method of obtaining the biological sample will vary depending on the type of biological sample being obtained, and such methods are well-known to those skilled in the art. For example, a sample of brain tissue can be obtained using a sterotactic brain needle biopsy, while a sample of cerebrospinal fluid can be obtained via a lumbar puncture.

### Alzheimer's Disease

In some embodiments, the imaging indicates a level of tau pathology sufficient to diagnose the subject as having AD. In further embodiments, the method indicates that the subject has early stage AD, an increased risk of developing AD, or both. A level of tau pathology sufficient to diagnose the subject as having AD or early stage AD can be due to the presence of increased levels of cell surface markers reflecting an increased level of tau phosphorylation (e.g., hyperphosphorylation) within the cell (e.g., neural cell). Examples of cell surface markers reflecting an increased level of tau phosphorylation include KRT6A, KRT6B, HSP, and VIM.

AD is a chronic neurodegenerative disease that usually starts slowly, gradually worsens over time, and is the cause of 60-70% of cases of dementia. AD is characterized by loss of neurons and synapses in the cerebral cortex and certain subcortical regions. This loss results in gross atrophy of the affected regions, including degeneration in the temporal lobe, parietal lobe, and parts of the frontal cortex and cingulate gyms. AD is a protein misfolding disease (proteopathy) caused by plaque accumulation of abnormally folded amyloid beta protein and tau protein in the brain.

Diagnosis of AD is most often made in the moderate stage. Typically, the symptoms of AD are cognitive dysfunction or deficiency and include dementia confirmed by medical and psychological exams, problems in at least two areas of mental functioning, and progressive loss of memory and other mental functions, especially where symptoms began between the ages of 40 and 90, no other disorders account for the dementia, and no other conditions are present that may mimic dementia, including hypothyroidism, overmedication, drug-drug interactions, vitamin B12 deficiency, and depression. As the disease advances, symptoms can include problems with language, disorientation (including easily getting lost), mood swings, loss of motivation, not managing self-care, and behavioral issues. In some embodiments, the methods and compositions described herein provide for the detection of early stage AD, which can be present before one or more of these symptoms has manifested. Accordingly, in some embodiments, the methods are used to diagnose a subject that does not exhibit any other symptoms of AD.

In some embodiments, the methods further comprise providing prophylaxis or treatment of AD to the subject. Prophylaxis of AD includes changes in lifestyle and diet that decrease the risk of developing AD. For example, intellectual activities such as reading, board games, solving puzzles, playing musical instruments, learning a second language, or even regular social interaction lead to a decreased risk of developing AD. Likewise, a healthy diet such as a Japanese or Mediterranean diet has been associated with decreasing the risk of developing AD.

Several medicines have also been identified that can be used to treat the cognitive problems associated with AD. These include acetylcholinesterase inhibitors such as tacrine, rivastigmine, galantamine, and donepezil, as well as the NMDA receptor antagonist memantine. Huperzine A is a promising agent for treating AD, and atypical antipsychotics can be used for reducing aggression and psychosis in people having AD.

### Pharmaceutical Compositions

In some embodiments, the compositions described herein are delivered as a pharmaceutical composition. Pharmaceutical compositions comprising the compositions of the invention are prepared according to standard techniques and further comprise a pharmaceutically acceptable carrier. Generally, normal saline will be employed as the pharmaceutically acceptable carrier. Other suitable carriers include, e.g., water, buffered water, isotonic solution (e.g., dextrose), 0.4% saline, 0.3% glycine, and the like, including glycoproteins for enhanced stability, such as albumin, lipoprotein, globulin, and the like. These compositions may be sterilized by conventional, well known sterilisation techniques. The resulting aqueous solutions may be packaged for use or filtered under aseptic conditions and lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents, and the like, for example, sodium acetate, sodium lactate, sodium chloride, potassium chloride, calcium chloride, and the like. Additionally, the liposome compositions of the invention can be suspended in suspensions that include lipid-protective agents that protect lipids against free-radical and lipid-peroxidative damages on storage. Lipophilic free-radical quenchers, such as α-tocopherol and water-soluble iron-specific chelators, such as ferrioxamine, are suitable.

The concentration of liposome compositions of the invention in the pharmaceutical formulations can vary widely, i.e., from less than about 0.05%, usually at or at least about 2-5%, to as much as 10 to 30% by weight, and will be selected primarily by fluid volumes, viscosities, etc., in accordance with the particular mode of administration selected. For example, the concentration may be increased to lower the fluid load associated with treatment. The amount of compositions administered will depend upon the particular aptamer used, the disease state being treated, and the judgment of the clinician. Generally, the amount of composition administered will be sufficient to deliver a therapeutically effective dose of the nucleic acid. The quantity of composition necessary to deliver a therapeutically effective dose can be determined by one skilled in the art. Typical dosages will generally be between about 0.01 and about 50 mg nucleic acid per kilogram of body weight, preferably between about 0.1 and about 10 mg nucleic acid/kg body weight, and most preferably between about 2.0 and about 5.0 mg nucleic acid/kg of body weight. For administration to mice, the dose is typically 50-100 μg per 20 g mouse.

### Kits

In some embodiments, the present invention also provides for kits for preparing the above-described liposome complexes/compositions. Such kits can be prepared from readily available materials and reagents, as described above. For example, such kits can comprise any one or more of the following materials: liposomes, nucleic acid (condensed or uncondensed), hydrophilic polymers, hydrophilic polymers derivatized with targeting ligands such as aptamers, and instructions. A wide variety of kits and components can be prepared, depending upon the intended user of the kit and the particular needs of the user. For example, the kit may contain any one of a number of targeting moieties for targeting the complex to a specific cell type, as described above.

Instructional materials for preparation and use of the liposome complexes can be included. While the instructional materials typically comprise written or printed materials, they are not limited to such. Any medium capable of storing such instructions and communicating them to an end user is contemplated. Such media include, but are not limited to, electronic storage media (e.g., magnetic discs, tapes, cartridges, chips), optical media (e.g., CD ROM), and the like. Such media may include addresses to internet sites that provide such instructional materials.

In various embodiments, the instructions may direct a user to carry out any of the method steps described herein. For example, the instructions may direct a user to diagnose the risk that a subject will develop AD by detecting the presence of tau pathology using the targeted liposomal compositions described herein.

Examples have been included to more clearly describe particular embodiments of the invention. However, there are a wide variety of other embodiments within the scope of the present invention, which should not be limited to the particular examples provided herein.

### EXAMPLES

### Example 1—Ligand and Target Identification

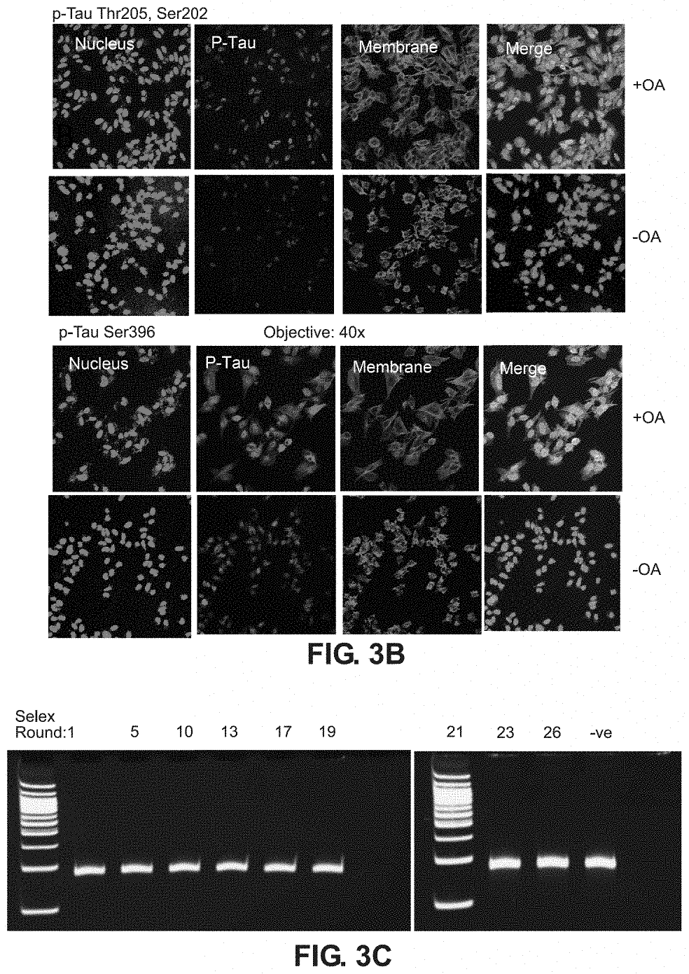

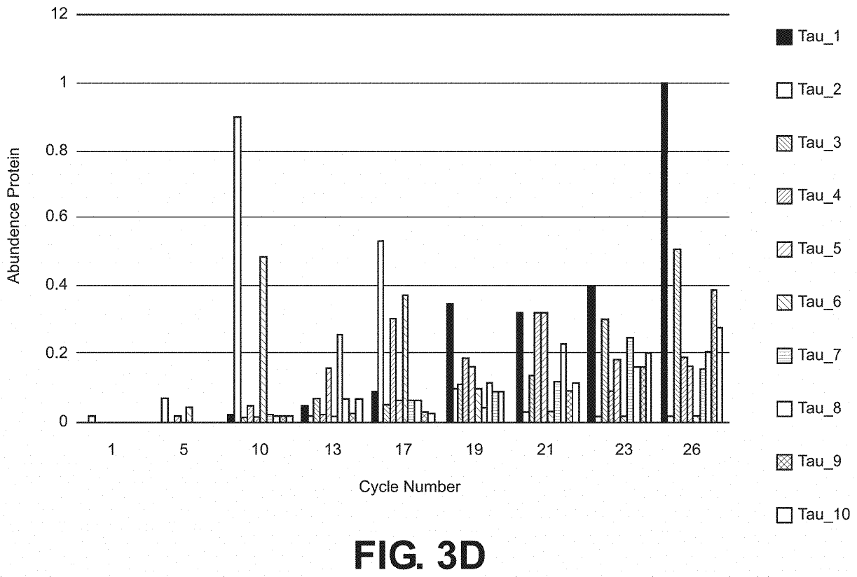

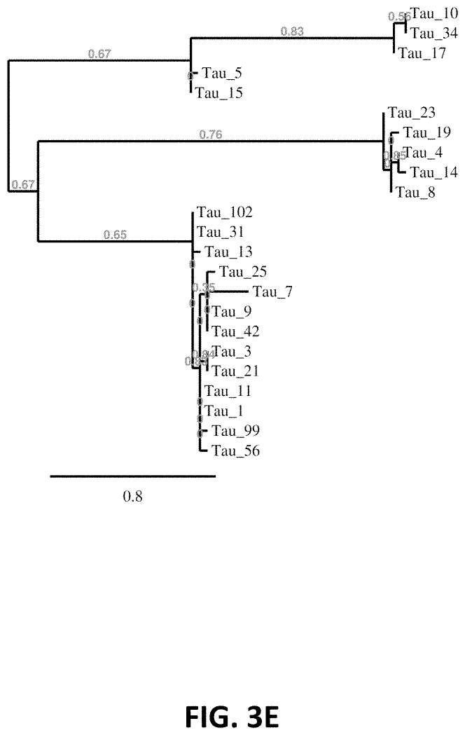

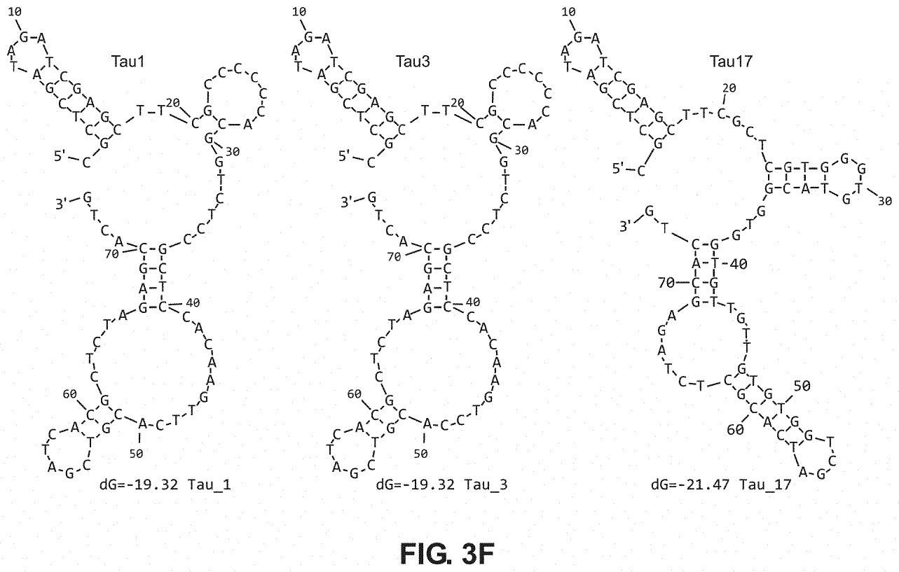

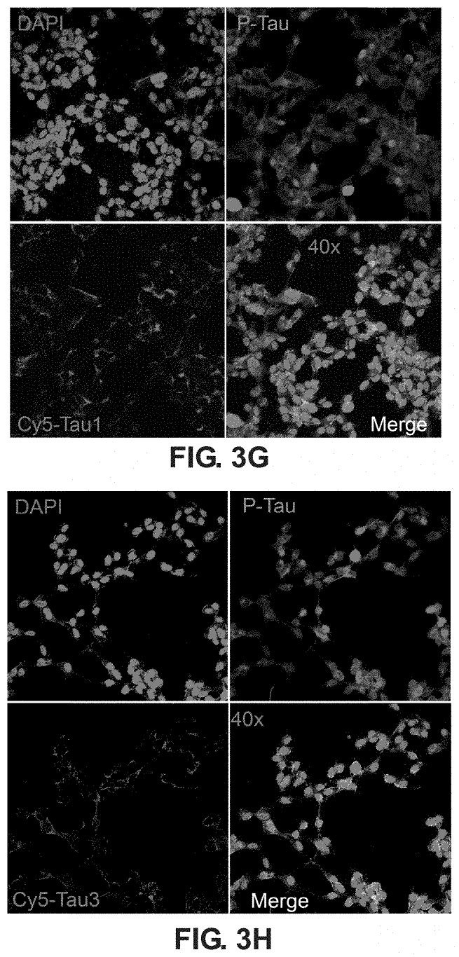

Elegant methods for the identification of cell-surface markers include the well-known phage display technique (Koivunen et al., J Biol Chem 268, 20205-20210 (1993)), and cell-SELEX: a method to screen DNA aptamers against cell borne targets. Shangguan, et al., Chembiochem 8, 603-606 (2007). The inventors have used cell-SELEX to identify aptamers that bind hyperphosphorylated SH-SYSY cells (a neuroblastoma cell line) differentiated to a neuronal phenotype. A summary of the screen and thioaptamer identification is shown in FIG. 3. Treatment of SH-SY5Y cells with retinoic acid induces a neuronal phenotype with axonal and neuritic structures (FIG. 3A). Treatment with okadaic acid, a potent inhibitor of PP2A then induces hyperphosphorylation, evidenced by nuclear pTau Thr205/Ser202 stained by the AT8 antibody (FIG. 3B, upper row), and cytosolic pTau Ser396 stained by the PHF-1 antibody (FIG. 3B, lower row).

Cell SELEX against the neuronal cells was conducted in “black-box” mode, isolating the cell membrane-binding thioaptamers by differential centrifugation and PCR amplifying them using primers specific to the leader sequences. The starting thioaptamer library was a 10¹⁵ member library that incorporated a 30 base random sequence bracketed by two primer regions (5′-GATATGTCTAGAGCCTCAGATCA-(N30)-CGGAGTTATGTTAGCAGTAGC-3′ SEQ ID NO: 28). Two negative SELEX steps were included at round 13 and round 21, comprising screening against cells treated with retinoic acid but not treated with okadaic acid, thus simulating “normal” or non-hyperphosphorylated neurons. In these steps, the supernatant, i.e. thioaptamers that did not bind to the cell membrane or become internalized, were isolated for amplification, thus insuring that the only thioaptamers continuing in the screen were those selectively binding hyperphosphorylated neurons. The top 250 sequences identified at cycle 26 are shown in Table 2, which is provided at the end of this Example 1. NextGen sequencing of the aptamers remaining at round 26 and at selected intermediate rounds (1, 5, 10, 13, 17, 19, 21, 23, 26) of Cell SELEX (FIG. 3C) was then conducted using the IonTorrent® method and the Ion318® chip, followed by sequence alignment using the Aptaligner code. Lu et al., Biochemistry 53, 3523-3525 (2014). The top 20 sequences fell into three distinct structural families (dendrogram in FIG. 3E). Evidence of the success of the negative-screen strategy is shown in FIG. 3D, where the thioaptamer Tau_2 (SEQ ID NO: 218) (orange bar) dominates the library at round 10, but after the negative screen at round 13, Tau_2 (SEQ ID NO: 218) practically disappears and is overtaken by e.g., Tau_1 (SEQ ID NO: 5) (blue bar). M-fold structures of Tau-1, Tau_3 (SEQ ID NO: 6), and Tau_17 (SEQ ID NO: 13) show remarkable similarity, consistent with their binding to a consistent, selective target. When synthesized as de novo sequences and exposed to hyperphosphorylated SH-SY5Y cells, Tau-1 and Tau_3 (SEQ ID NO: 6) bound avidly to the membrane and axonal processes (FIGS. 3G& 3H).

The protein target(s) of thioaptamers Tau_1 (SEQ ID NO: 5), Tau_3 (SEQ ID NO: 6), Tau_4 (SEQ ID NO: 12), and Tau_5 (SEQ ID NO: 14), were identified by affinity-pull down using the thioaptamers as the capturing reagent, followed by mass-spectroscopy. A scrambled DNA sequence, R4, was used as the control. The hyperphosphorylated, neuronally-transformed SH-SY5Y cells, at 90-95% confluence, were washed with cold PBS buffer and were incubated with biotinylated thioaptamers and R4 (24 mM each) individually at 4° C., in PBS (Dulbecco's PBS with calcium chloride and magnesium chloride) with gentle agitation for 2 hours. After incubation, the cells were cross-linked with 1% formaldehyde for 10 minutes at room temperature. The formaldehyde cross-linking was quenched with glycine. Cells were scraped from the flask, washed, lysed with lysing buffer, and treated with protease inhibitor mixture. The lysates were freeze-thawed for 30 minutes on ice and cleared by centrifuging at 10,000 g for 2 min at 4° C.

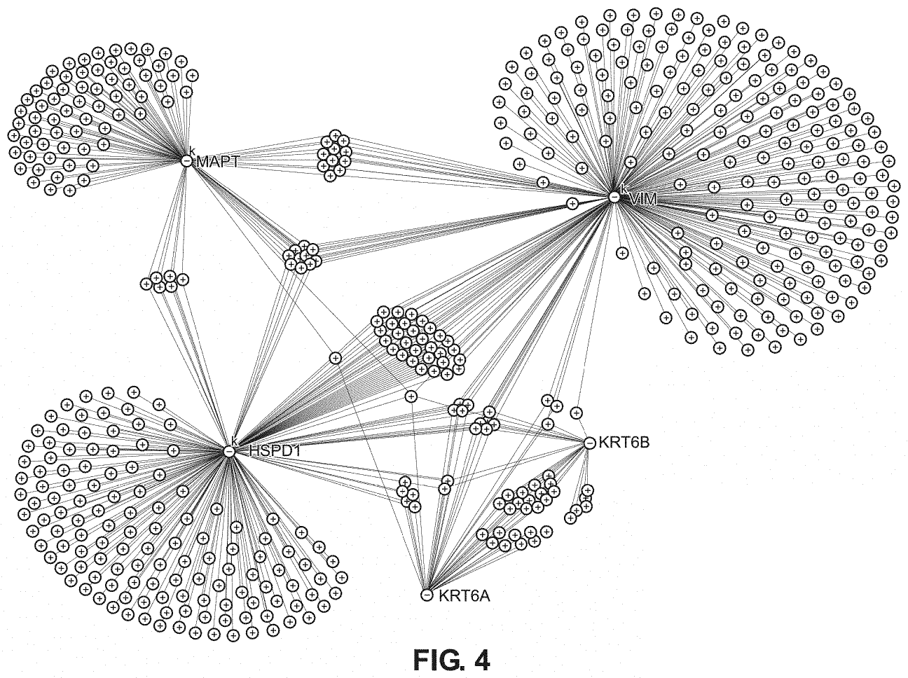

To pull down the cross-linked proteins, equal amounts of cell lysate were incubated with pre-washed streptavidin magnetic beads for 1 hour at room temperature with continuous rotation. Protein digestions were performed on the beads to isolate targeted protein(s), and samples were processed for mass spectrometric analysis. Each sample was analyzed in triplicate. The raw data files were processed to generate a Mascot Generic Format with Mascot Distiller and searched against the SwissProt 2012_01 (Human) database using the Mascot search engine v2.3.02 run on an in-house server. Proteins that were present in the control (R4) pulldown were disregarded in the test thioaptamer pulldowns. Those that remained were considered as unique hits. Thus, Tau_1 (SEQ ID NO: 5) showed avid binding to HSPD1 (emPAI>6), and KRT6A/KRT6B (emPAI˜0.38 each). Tau_3 (SEQ ID NO: 6) showed avid binding to VIM (emPAI=2.7) and HSPD1 (emPAI˜0.62). Tau_4 (SEQ ID NO: 12) showed binding to HSPD1 (emPAI˜1.08) and KRT6A (emPAI˜0.79). The VisANT database yielded connections between each of these and tau, as shown in FIG. 4. The identification of HSP is an indirect confirmation that the cell model is indeed causing misfolding. VIM redistribution in the cytoskeleton and membrane has been described collateral to tau aggresome formation. However, no mechanistic studies have been conducted. There have been associations of Keratin 9 with tau pathology, but not KRT6A/KRT6B.

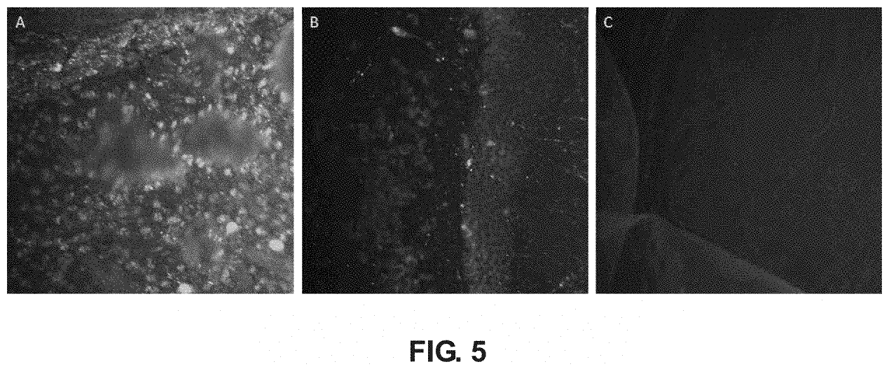

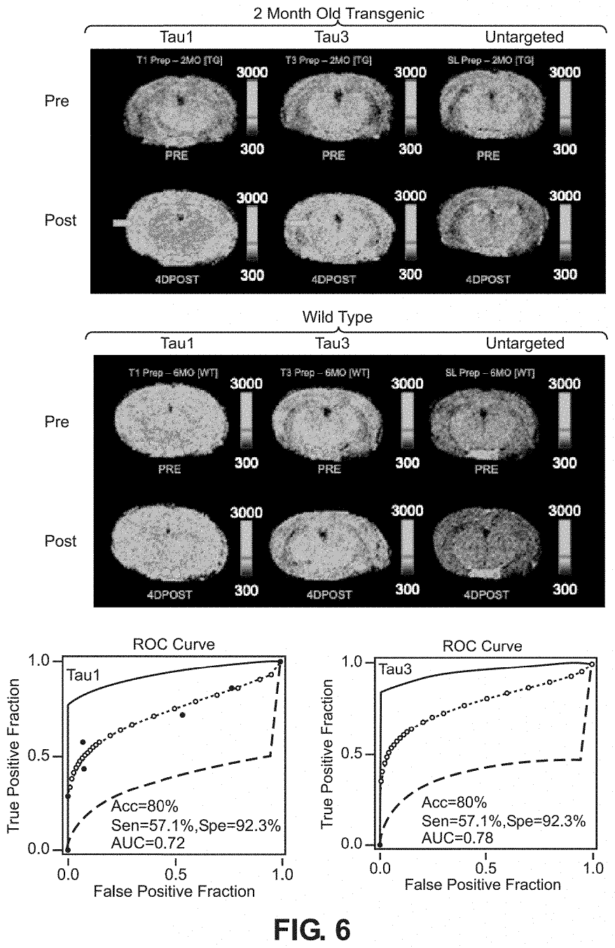

The thioaptamers Tau_1 (SEQ ID NO: 5), Tau_3 (SEQ ID NO: 6), Tau_4 (SEQ ID NO: 12), and Tau_5 (SEQ ID NO: 14) were each individually synthesized with a 3′ Cy3 tag and incubated with P301S mouse brain tissue, which was then counterstained with one of the pTau antibodies, AT100 (indicative of late stage phosphorylation) or AT8 (indicative of early stage phosphorylation). Tau_3 (SEQ ID NO: 6) showed the strongest staining and bound the hippocampal tissue in a manner highly correlated with the AT100 antibody (FIG. 5A), but did not correlate with AT8 (FIG. 5B), and did not stain normal brain tissue (FIG. 5C). Thus, Tau_3 (SEQ ID NO: 6) is a suitable marker of tau phosphorylation.

TABLE 2
Top 250 aptamer sequence from SELEX Cycle 26
(the numerical aspect of the identifier in Table 2 correlates
with the numerical aspect of the identifier in Table 1)

| Identifier | Nucleotide Sequence | SEQ ID NO |
|---|---|---|
| Seq_164 | CTTTGACCCAAACACAACTGCGGTGAATCC | SEQ ID NO: 29 |
| Seq_241 | CTCCAACCTGGACCCCAAACGAACTGAGAT | SEQ ID NO: 30 |
| Seq_168 | CACGTCAACCACACCAAATTGGGGACCGAA | SEQ ID NO: 31 |
| Seq_213 | CTCGTACGTCAACCTCACCAAATTGGGAAC | SEQ ID NO: 32 |
| Seq_112 | CTCGCACGTCAACCACACCAAATTGGGGAC | SEQ ID NO: 33 |
| Seq_225 | CTCGTACGTCAACCACACCAAATTGGGGAC | SEQ ID NO: 34 |
| Seq_4 | CTCGTCCCACCACAACATTATCTCAACGCC | SEQ ID NO: 12 |
| Seq_8 | CTCGTCCCACCACAACATCATCTCAACGCC | SEQ ID NO: 11 |
| Seq_18 | CTCGTCTCACCACAACATTATCTCAACGCC | SEQ ID NO: 35 |
| Seq_50 | CTCGTCTCACCACAACATCATCTCAACGCC | SEQ ID NO: 36 |
| Seq_12 | CTCGCCTCACCACAACATTATCTCAACGCC | SEQ ID NO: 37 |
| Seq_16 | CTCGCCCCACCACAACATTATCTCAACGCC | SEQ ID NO: 38 |
| Seq_23 | CTCGCCCCACCACAACATCATCTCAACGCC | SEQ ID NO: 25 |
| Seq_27 | CTCGCCTCACCACAACATCATCTCAACGCC | SEQ ID NO: 39 |
| Seq_181 | CTCGTCTCACCATAACATTATCTCAACGCC | SEQ ID NO: 40 |
| Seq_96 | CTCGTCCCACCATAACATTATCTCAACGCC | SEQ ID NO: 41 |
| Seq_101 | CTCGTCCCACCATAACATCATCTCAACGCC | SEQ ID NO: 42 |
| Seq_110 | CTCGCCTCACCATAACATTATCTCAACGCC | SEQ ID NO: 43 |
| Seq_174 | CTCGCCCCACCATAACATTATCTCAACGCC | SEQ ID NO: 44 |
| Seq_198 | CTCGCCCCACCATAACATCATCTCAACGCC | SEQ ID NO: 45 |
| Seq_210 | CTCGCCTCACCATAACATCATCTCAACGCC | SEQ ID NO: 46 |
| Seq_120 | CTCGCCTCACCACAACATTATCCCAACGCC | SEQ ID NO: 47 |
| Seq_160 | CTCGCCCCACCACAACATTATCCCAACGCC | SEQ ID NO: 48 |
| Seq_170 | CTCGCCTCACCACAACATTGTCCCAACGCC | SEQ ID NO: 49 |
| Seq_244 | CTCGCCCCACCACAACATTGTCCCAACGCC | SEQ ID NO: 50 |
| Seq_175 | CTCGTCCCACCACAACATTGTCTCAATGCC | SEQ ID NO: 51 |
| Seq_243 | CTCGCCCCACCACAACATTGTCTCAATGCC | SEQ ID NO: 52 |
| Seq_14 | CTCGTCCCACCACAACATTGTCTCAACGCC | SEQ ID NO: 20 |
| Seq_37 | CTCGCCCCACCACAACATTGTCTCAACGCC | SEQ ID NO: 53 |
| Seq_46 | CTCGCCTCACCACAACATTGTCTCAACGCC | SEQ ID NO: 54 |
| Seq_121 | CTCGTCTCACCACAACATTGTCTCAACGCC | SEQ ID NO: 55 |
| Seq_224 | CTCGTCCCACCACAACATTGCCTCAACGCC | SEQ ID NO: 56 |
| Seq_78 | CTCGTCCCACCACAACATCGTCTCAACGCC | SEQ ID NO: 57 |
| Seq_165 | CTCGTCCCACCATAACATTGTCTCAACGCC | SEQ ID NO: 58 |
| Seq_242 | CTCGTCCCACCACAACACTATCCCAACGCC | SEQ ID NO: 59 |
| Seq_212 | CTCGTCTCACCACAACATTATCCCAACGCC | SEQ ID NO: 60 |
| Seq_183 | CTCGTCCCACCACAACATCATCCCAACGCC | SEQ ID NO: 61 |
| Seq_64 | CTCGTCCCACCACAACATTATCCCAACGCC | SEQ ID NO: 62 |
| Seq_115 | CTCGTCCCACCACAACATTGTCCCAACGCC | SEQ ID NO: 63 |
| Seq_232 | CTCGTCCCACCACAACAATATCTCAACGCC | SEQ ID NO: 64 |
| Seq_196 | CTCGTCCCACCACAACATTATCTCGACGCC | SEQ ID NO: 65 |
| Seq_201 | CTCGTCCCACCGCAACATTATCTCAACGCC | SEQ ID NO: 66 |
| Seq_233 | CTCGCCTCACCACAACATTATCTCAATGCC | SEQ ID NO: 67 |
| Seq_238 | CTCGCCTCACCACAACATCATCTCAATGCC | SEQ ID NO: 68 |
| Seq_57 | CTCGTCCCACCACAACATCATCTCAATGCC | SEQ ID NO: 69 |
| Seq_61 | CTCGTCCCACCACAACATTATCTCAATGCC | SEQ ID NO: 70 |
| Seq_85 | CTCGTCCCACCACAACACCATCTCAATGCC | SEQ ID NO: 71 |
| Seq_184 | CTCGTCCCACCACAACACTATCTCAATGCC | SEQ ID NO: 72 |
| Seq_205 | CTCGCCCCACCACAACACCATCTCAATGCC | SEQ ID NO: 73 |
| Seq_98 | CTCGCCCCACCACAACATTATCTCAATGCC | SEQ ID NO: 74 |
| Seq_142 | CTCGCCCCACCACAACATCATCTCAATGCC | SEQ ID NO: 75 |
| Seq_151 | CTCGTCCCACCACAACACCACCTCAACGCC | SEQ ID NO: 76 |
| Seq_189 | CTCGTCCCACCACAACATCACCTCAACGCC | SEQ ID NO: 77 |
| Seq_190 | CTCGTCCCACCATAACACCATCTCAACGCC | SEQ ID NO: 78 |
| Seq_228 | CTCGTCCCACCATAACACTATCTCAACGCC | SEQ ID NO: 79 |
| Seq_117 | CTCGTCTCACCACAACACTATCTCAACGCC | SEQ ID NO: 80 |
| Seq_134 | CTCGTCTCACCACAACACCATCTCAACGCC | SEQ ID NO: 81 |
| Seq_19 | CTCGTCCCACCACAACACCATCTCAACGCC | SEQ ID NO: 21 |
| Seq_24 | CTCGTCCCACCACAACACTATCTCAACGCC | SEQ ID NO: 82 |
| Seq_51 | CTCGTCCCACCACAACACTGTCTCAACGCC | SEQ ID NO: 83 |
| Seq_70 | CTCGTCCCACCACAACACCGTCTCAACGCC | SEQ ID NO: 84 |
| Seq_157 | CTCGTCCCACCACAGCATTATCTCAACGCC | SEQ ID NO: 85 |
| Seq_191 | CTCGTCCCACCACAGCATCATCTCAACGCC | SEQ ID NO: 86 |
| Seq_119 | CTCGTCCCACCACAACATTATCTCAGCGCC | SEQ ID NO: 87 |
| Seq_145 | CTCGTCCCACCACAACATCATCTCAGCGCC | SEQ ID NO: 88 |
| Seq_139 | CTCGTCCCGCCACAACATTATCTCAACGCC | SEQ ID NO: 89 |
| Seq_237 | CTCGTCCCGCCACAACATCATCTCAACGCC | SEQ ID NO: 90 |
| Seq_177 | CTCGCCTCACCACAACACAATCTCAATGCC | SEQ ID NO: 91 |
| Seq_178 | CTCGCCTCACCATAACACAATCTCAACGCC | SEQ ID NO: 92 |
| Seq_93 | CTCGCCTCACCACAACACTATCTCAACGCC | SEQ ID NO: 93 |
| Seq_53 | CTCGCCTCACCACAACACAATCTCAACGCC | SEQ ID NO: 94 |
| Seq_65 | CTCGCCTCACCACAACACCATCTCAACGCC | SEQ ID NO: 95 |
| Seq_100 | CTCGCCCCACCACAACACTATCTCAACGCC | SEQ ID NO: 96 |
| Seq_63 | CTCGCCCCACCACAACACCATCTCAACGCC | SEQ ID NO: 97 |
| Seq_95 | CTCGCCCCACCACAACACAATCTCAACGCC | SEQ ID NO: 98 |
| Seq_144 | CTCGCCCCACCACAACACTGTCTCAACGCC | SEQ ID NO: 99 |
| Seq_156 | CTCGCCTCACCACAACACTGTCTCAACGCC | SEQ ID NO: 100 |
| Seq_229 | CTCGCCTCACCACAACACCGTCTCAACGCC | SEQ ID NO: 101 |
| Seq_240 | CTCGCCCCACCACAACACCGTCTCAACGCC | SEQ ID NO: 102 |
| Seq_250 | CTCGTGTCCACACCATTCACAACGCCAAAT | SEQ ID NO: 103 |
| Seq_230 | CCTCACCACAACATTGTCTCAACGCCACAA | SEQ ID NO: 104 |
| Seq_52 | TCCCACCACAACATTGTCTCAACGCCACAA | SEQ ID NO: 105 |
| Seq_169 | CCCCACCACAACATTGTCTCAACGCCACAA | SEQ ID NO: 106 |
| Seq_173 | TCCCACCACAACACTGTCTCAACGCCACAA | SEQ ID NO: 107 |
| Seq_239 | TCCCACCACAACACCGTCTCAACGCCACAA | SEQ ID NO: 108 |
| Seq_22 | TCCCACCACAACATTATCTCAACGCCACAA | SEQ ID NO: 109 |
| Seq_30 | TCCCACCACAACATCATCTCAACGCCACAA | SEQ ID NO: 110 |
| Seq_74 | TCCCACCACAACACCATCTCAACGCCACAA | SEQ ID NO: 111 |
| Seq_136 | TCCCACCACAACACTATCTCAACGCCACAA | SEQ ID NO: 112 |
| Seq_216 | TCCCACCACAACATTATCTCAACGCCACAG | SEQ ID NO: 113 |
| Seq_227 | TCCCACCACAACATCATCTCAACGCCATAA | SEQ ID NO: 114 |
| Seq_137 | TCCCACCACAACATTATCTCAACGCCATAA | SEQ ID NO: 115 |
| Seq_222 | TCCCACCACAACATTGTCTCAACGCCATAA | SEQ ID NO: 116 |
| Seq_211 | TCCCACCACAACATCATCTCAATGCCACAA | SEQ ID NO: 117 |
| Seq_235 | TCCCACCACAACACCATCTCAATGCCACAA | SEQ ID NO: 118 |
| Seq_83 | CCCCACCACAACATTATCTCAACGCCACAA | SEQ ID NO: 119 |
| Seq_128 | CCCCACCACAACATCATCTCAACGCCACAA | SEQ ID NO: 120 |
| Seq_143 | CCTCACCACAACATTATCTCAACGCCACAA | SEQ ID NO: 121 |
| Seq_172 | CCTCACCACAACATCATCTCAACGCCACAA | SEQ ID NO: 122 |
| Seq_220 | CCCCACCACAACACCATCTCAACGCCACAA | SEQ ID NO: 123 |
| Seq_231 | CCTCACCACAACACAATCTCAACGCCACAA | SEQ ID NO: 124 |
| Seq_206 | GCGCCGCCTACGCACAACCAATCACACCAT | SEQ ID NO: 125 |
| Seq_209 | GCGCCGCCTACGCACAACCAATCACACCAC | SEQ ID NO: 126 |
| Seq_140 | CCCACAGCACCAACACACACACCCGTATAA | SEQ ID NO: 127 |
| Seq_153 | CCCACAGCACCAACACACACATCCGTATAA | SEQ ID NO: 128 |
| Seq_155 | CTCGCCCACAGCACCAACACACATACCCGT | SEQ ID NO: 129 |
| Seq_29 | CTCGCCCACAGCACCAACACACACACCCGT | SEQ ID NO: 130 |
| Seq_48 | CTCGCCCACAGCACCAACACACACATCCGT | SEQ ID NO: 131 |
| Seq_135 | CTCGCCCACAGCACCAACACACACATCCGC | SEQ ID NO: 132 |
| Seq_159 | CTCGCCCACAGCACCAACACACACACCCGC | SEQ ID NO: 133 |
| Seq_125 | CTCCGACACCACGAGGAGATGCACCTGCAA | SEQ ID NO: 134 |
| Seq_217 | CTCGAACACACCCAGGGAATACACGAAACA | SEQ ID NO: 135 |
| Seq_248 | CTCCCCACAGTCTCCACCCCACAAGCCCAC | SEQ ID NO: 136 |
| Seq_200 | CTCCCCACAGTCTCCATTCCACAAGCTCAC | SEQ ID NO: 137 |
| Seq_202 | CTCCCCACAGTCTCCACTCCACAAGCTCAC | SEQ ID NO: 138 |
| Seq_163 | CTCCCCACAGTCTCCATTCCACAGGCCCAC | SEQ ID NO: 139 |
| Seq_226 | CTCCCCACAGTCTCCACTCCACAGGCCCAC | SEQ ID NO: 140 |
| Seq_44 | CTCCCCACAGTCTCCACTCCACAAGCCCAC | SEQ ID NO: 141 |
| Seq_49 | CTCCCCACAGTCTCCATTCCACAAGCCCAC | SEQ ID NO: 142 |
| Seq_111 | CTCCCCACAGTCCCCACTCCACAAGCCCAC | SEQ ID NO: 143 |
| Seq_123 | CTCCCCACAGTCCCCATTCCACAAGCCCAC | SEQ ID NO: 144 |
| Seq_107 | CTCCCCACAGCCTCCACTCCACAAGTCCAC | SEQ ID NO: 145 |
| Seq_167 | CTCCCCACAGCCTCCACTCCACAAGCCCAC | SEQ ID NO: 146 |
| Seq_249 | CTCCCCACAGCCTCCATTCCACAAGCTCAC | SEQ ID NO: 147 |
| Seq_108 | CTCCCCACAGCCTCCATTCCACAAGCCCAC | SEQ ID NO: 148 |
| Seq_214 | CTCCCCACAGCCCCCATTCCACAAGCCCAC | SEQ ID NO: 149 |
| Seq_32 | CTCCCCACAGTCTCCACTCCACAAGTTCAC | SEQ ID NO: 150 |
| Seq_35 | CTCCCCACAGTCCCCACTCCACAAGTTCAC | SEQ ID NO: 151 |
| Seq_40 | CTCCCCACAGTCTCCATTCCACAAGTTCAC | SEQ ID NO: 152 |
| Seq_68 | CTCCCCACAGTCCCCATTCCACAAGTTCAC | SEQ ID NO: 153 |
| Seq_38 | CTCCCCACAGCCTCCATTCCACAAGTTCAC | SEQ ID NO: 154 |
| Seq_39 | CTCCCCACAGCCTCCACTCCACAAGTTCAC | SEQ ID NO: 155 |
| Seq_72 | CTCCCCACAGCCCCCACTCCACAAGTTCAC | SEQ ID NO: 156 |
| Seq_89 | CTCCCCACAGCCCCCATTCCACAAGTTCAC | SEQ ID NO: 157 |
| Seq_176 | CTCCCCACAGCCCCCACTCCACAAGTCCAC | SEQ ID NO: 158 |
| Seq_60 | CTCCCCACAGTCTCCACTCCACAAGTCCAC | SEQ ID NO: 159 |
| Seq_77 | CTCCCCACAGTCCCCACTCCACAAGTCCAC | SEQ ID NO: 160 |
| Seq_86 | CTCCCCACAGCCTCCATTCCACAAGTCCAC | SEQ ID NO: 161 |
| Seq_92 | CTCCCCACAGTCTCCATTCCACAAGTCCAC | SEQ ID NO: 162 |
| Seq_129 | CTCCCCACAGTCCCCATTCCACAAGTCCAC | SEQ ID NO: 163 |
| Seq_180 | CTCCCCACAGCCCCCATTCCACAAGTCCAC | SEQ ID NO: 164 |
| Seq_150 | CTCCCCACAGTCTCCACCCCACAAGTTCAC | SEQ ID NO: 165 |
| Seq_194 | CTCCCCACAGCCTCCACCCCACAAGTTCAC | SEQ ID NO: 166 |
| Seq_207 | CTCCCCACAGTCCCCACTCCACAGGTTCAC | SEQ ID NO: 167 |
| Seq_146 | CTCCCCACAGTCTCCACTCCACAGGTTCAC | SEQ ID NO: 168 |
| Seq_158 | CTCCCCACAGTCTCCATTCCACAGGTTCAC | SEQ ID NO: 169 |
| Seq_171 | CTCCCCACAGCCTCCATTCCACAGGTTCAC | SEQ ID NO: 170 |
| Seq_215 | CTCCCCACAGCCTCCACTCCACAGGTTCAC | SEQ ID NO: 171 |
| Seq_105 | CCCCCCATTGGCTCCGCTCCACACAGCTTC | SEQ ID NO: 172 |
| Seq_185 | CCCCCATTGGCTCCGCTCCACACGGCTTCA | SEQ ID NO: 173 |
| Seq_161 | CCCCCATTGGCTCCGCTCCACACAACTTCA | SEQ ID NO: 174 |
| Seq_7 | CCCCCATTGGCTCCGCTCCACACAGCTTCA | SEQ ID NO: 17 |
| Seq_62 | CCCCCATTGGCTCCGCTCCACACAGCCTCA | SEQ ID NO: 175 |
| Seq_197 | CCCCCCCGCGGTCTCCGCTCCACAAGTTCA | SEQ ID NO: 176 |
| Seq_71 | CCCCCCCACGGTCTCCGCTCCACAAGCCCA | SEQ ID NO: 177 |
| Seq_102 | CCCCCCCACGGTCTCCGCTCCACAAGCTCA | SEQ ID NO: 27 |
| Seq_11 | CCCCCCCACGGTCTCCGCTCCACAAGTTCA | SEQ ID NO: 8 |
| Seq_21 | CCCCCCCACGGTCTCCGCTCCACAAGTCCA | SEQ ID NO: 15 |
| Seq_42 | CCCCCCCACGGTCTCCGCTCCACAGGTTCA | SEQ ID NO: 19 |
| Seq_141 | CCCCCCCACGGTCTCCGCTCCACAGGTCCA | SEQ ID NO: 178 |
| Seq_104 | CCCCCACGGTCTCCGCTCCACAAGTTCACA | SEQ ID NO: 179 |
| Seq_193 | CCCCCACGGTCTCCGCTCCACAAGTCCACA | SEQ ID NO: 180 |
| Seq_l | CCCCCCACGGTCTCCGCTCCACAAGTTCAC | SEQ ID NO: 5 |
| Seq_3 | CCCCCCACGGTCTCCGCTCCACAAGTCCAC | SEQ ID NO: 6 |
| Seq_9 | CCCCCCACGGTCTCCGCTCCACAGGTTCAC | SEQ ID NO: 7 |
| Seq_25 | CCCCCCACGGTCTCCGCTCCACAGGTCCAC | SEQ ID NO: 16 |
| Seq_182 | CCCCCCACGGTCTCCGCTCCACAAGCACAC | SEQ ID NO: 181 |
| Seq_13 | CCCCCCACGGTCTCCGCTCCACAAGCCCAC | SEQ ID NO: 10 |
| Seq_31 | CCCCCCACGGTCTCCGCTCCACAAGCTCAC | SEQ ID NO: 18 |
| Seq_69 | CCCCCCACGGTCTCCGCTCCACAGGCCCAC | SEQ ID NO: 182 |
| Seq_148 | CCCCCCACGGTCTCCGCTCCACAGGCTCAC | SEQ ID NO: 183 |
| Seq_99 | CCCCCCACGGTCTCCGCTCCACAAGTTCGC | SEQ ID NO: 26 |
| Seq_122 | CCCCCCACGGTCTCCGCTCCACAAGTCCGC | SEQ ID NO: 184 |
| Seq_75 | CCCCCCACGGCCTCCGCTCCACAAGTTCAC | SEQ ID NO: 185 |
| Seq_186 | CCCCCCACGGCCTCCGCTCCACAAGTCCAC | SEQ ID NO: 186 |
| Seq_221 | CCCCCCCCGGTCTCCGCTCCACAAGTTCAC | SEQ ID NO: 187 |
| Seq_56 | CCCCCCGCGGTCTCCGCTCCACAAGTTCAC | SEQ ID NO: 23 |
| Seq_131 | CCCCCCGCGGTCTCCGCTCCACAAGTCCAC | SEQ ID NO: 188 |
| Seq_36 | CTCTCTGGTCCCCCCGGCCGTCCCTCTCAT | SEQ ID NO: 189 |
| Seq_236 | CTCGTACCACCCCCGGCCGTCCCTCTCATC | SEQ ID NO: 190 |
| Seq_130 | CTCCCCGTCCACCTCGCACCCAAGGCAATC | SEQ ID NO: 191 |
| Seq_26 | CTCCCCGTCCACCTCGCACTCAAGGCAATC | SEQ ID NO: 192 |
| Seq_109 | CTCCCCGTCCACCCCGCACTCAAGGCAATC | SEQ ID NO: 193 |
| Seq_80 | CTCCCCCCCACCTGGCACTGTCCCCGGAGA | SEQ ID NO: 194 |
| Seq_154 | CTCCCCACCTGGCACTGTCCCAACGCCACA | SEQ ID NO: 195 |
| Seq_246 | CTCGGCCAGCAGTTACAGCACACCACACTT | SEQ ID NO: 196 |
| Seq_162 | CTCCGACGGGATGTTCGACGAGCACACACT | SEQ ID NO: 197 |
| Seq_218 | CTCCGACGGGGTGTTCGACGAGCACACACT | SEQ ID NO: 198 |
| Seq_166 | CCCCGACGGGATGTTCGATGAGCACACACT | SEQ ID NO: 199 |
| Seq_97 | CTCCGACGGGATGTTCGATGAGCACACACC | SEQ ID NO: 200 |
| Seq_5 | CTCCGACGGGATGTTCGATGAGCACACACT | SEQ ID NO: 14 |
| Seq_15 | CTCCGACGGGGTGTTCGATGAGCACACACT | SEQ ID NO: 22 |
| Seq_204 | TGCCCTCCGCTCGTATTGTCACCCCGCAATG | SEQ ID NO: 201 |
| Seq_114 | CGCCTGCTGCCTTCCCATACGTCGATCCAG | SEQ ID NO: 202 |
| Seq_195 | CGCCTGCTGCCTTCCCACACGTCGATCCAG | SEQ ID NO: 203 |
| Seq_43 | CGCCTGCTGCCTTCCTATACGCCGATCCAG | SEQ ID NO: 204 |
| Seq_81 | CGCCTGCTGCCTTCCTATACGTCGATCCAG | SEQ ID NO: 205 |
| Seq_55 | CGCCTGCTGCCTTCCTGTACGTCGATCCAG | SEQ ID NO: 206 |
| Seq_133 | CGCCTGCTGCCTTCCTGTACGCCGATCCAG | SEQ ID NO: 207 |
| Seq_138 | CTCGCTGACCAGATGAGGGGGGTTTACTGG | SEQ ID NO: 208 |
| Seq_208 | CTCGCTGACCAGATGAAGGGGGTTTACTGG | SEQ ID NO: 209 |
| Seq_203 | CTCGCCGACCAGATGAAGGGGGGTTTACTG | SEQ ID NO: 210 |
| Seq_127 | CTCGCTGACCAGATGGAGGGGGGTTTACTG | SEQ ID NO: 211 |
| Seq_91 | CTCGCTGACCAGGTGAAGGGGGGTTTACTG | SEQ ID NO: 212 |
| Seq_90 | CTCGCTGGCCAGATGAAGGGGGGTTTACTG | SEQ ID NO: 213 |
| Seq_76 | CTCGCTGACCGGATGAAGGGGGGTTTACTG | SEQ ID NO: 214 |
| Seq_67 | CTCGCTGACCAGACGAAGGGGGGTTTACTG | SEQ ID NO: 215 |
| Seq_66 | CTCGCTGACCAGATGAAGGGGGGTTTGCTG | SEQ ID NO: 216 |
| Seq_54 | CTCGCTGACCAGATGAGGGGGGGTTTACTG | SEQ ID NO: 217 |
| Seq_2 | CTCGCTGACCAGATGAAGGGGGGTTTACTG | SEQ ID NO: 218 |
| Seq_47 | CTCGCTGACCAGATGAAGGGGGGCTTACTG | SEQ ID NO: 219 |
| Seq_59 | CTGACCAGATGAAGGGGGGGTTTACTGGGG | SEQ ID NO: 220 |
| Seq_199 | CTGACCAGATGGAGGGGGGTTTACTGGGGG | SEQ ID NO: 221 |
| Seq_188 | CCGACCAGATGAAGGGGGGTTTACTGGGGG | SEQ ID NO: 222 |
| Seq_179 | CTGGCCAGATGAAGGGGGGTTTACTGGGGG | SEQ ID NO: 223 |
| Seq_147 | CTGACCAGGTGAAGGGGGGTTTACTGGGGG | SEQ ID NO: 224 |
| Seq_132 | CTGACCGGATGAAGGGGGGTTTACTGGGGG | SEQ ID NO: 225 |
| Seq_118 | CTGACCAGATGAGGGGGGGTTTACTGGGGG | SEQ ID NO: 226 |
| Seq_106 | CTGACCAGATGAAGGGGGGTTTGCTGGGGG | SEQ ID NO: 227 |
| Seq_6 | CTGACCAGATGAAGGGGGGTTTACTGGGGG | SEQ ID NO: 228 |
| Seq_82 | CTGACCAGATGAAGGGGGGCTTACTGGGGG | SEQ ID NO: 229 |
| Seq_20 | GTGGGTGTGTATGTGTGGCGGGGGTGCGTT | SEQ ID NO: 230 |
| Seq_88 | GGTGTATTCTCCGTGGCGGGGGTGCGTTGG | SEQ ID NO: 231 |
| Seq_126 | TTGGGTGTATTCTCCGTGGCGGGGTGCGTT | SEQ ID NO: 232 |
| Seq_33 | CTCGGGTTCATGTGTTGTGTGGGTGGGGGT | SEQ ID NO: 233 |
| Seq_58 | GGTTCATGTGTTGTGTGGGTGGGGGTGTGT | SEQ ID NO: 234 |
| Seq_103 | CTCGGTGTCCAGATTGATGTTGGGGTGGGG | SEQ ID NO: 235 |
| Seq_234 | TGGGTGTGCGGTGGTGTTGTTGTGTGGGTG | SEQ ID NO: 236 |
| Seq_34 | TGGGTGTGTGGTGGTGTTGTTGTGTGGGTG | SEQ ID NO: 24 |
| Seq_94 | TGGGTGTGTGGTGGTGTTGTTGTGTGGATG | SEQ ID NO: 237 |
| Seq_41 | TGGGTGTACGGTGGTGTTGTTGTGTGGGTG | SEQ ID NO: 238 |
| Seq_73 | TGGGTATACGGTGGTGTTGTTGTGTGGGTG | SEQ ID NO: 239 |
| Seq_116 | TGGGTGTACGGTAGTGTTGTTGTGTGGGTG | SEQ ID NO: 240 |
| Seq_187 | TGGGTGTACGGTTGTGTTGTTGTGTGGGTG | SEQ ID NO: 241 |
| Seq_247 | CTCGTGGGTATGCGGTGGTGTTGTTGTGTG | SEQ ID NO: 242 |
| Seq_219 | CTCGTGGGTGTATGGTGGTGTTGTTGTGTG | SEQ ID NO: 243 |
| Seq_149 | CTCGCGGGTGTGTGGTGGTGTTGTTGTGTG | SEQ ID NO: 244 |
| Seq_124 | CTCGTGGGTGTGTGGTAGTGTTGTTGTGTG | SEQ ID NO: 245 |
| Seq_152 | CTCGTGGGTGTGTGGTTGTGTTGTTGTGTG | SEQ ID NO: 246 |
| Seq_87 | CTCGTGGGTGTGTGGTGGTGCTGTTGTGTG | SEQ ID NO: 247 |
| Seq_10 | CTCGTGGGTGTGTGGTGGTGTTGTTGTGTG | SEQ ID NO: 9 |
| Seq_84 | CTCGTGGGTGTGCGGTGGTGTTGTTGTGTG | SEQ ID NO: 248 |
| Seq_113 | CTCGTGGGTATACGGTAGTGTTGTTGTGTG | SEQ ID NO: 249 |
| Seq_223 | CTCGTGGGTATACGGTTGTGTTGTTGTGTG | SEQ ID NO: 250 |
| Seq_17 | CTCGTGGGTGTACGGTGGTGTTGTTGTGTG | SEQ ID NO: 13 |
| Seq_28 | CTCGTGGGTATACGGTGGTGTTGTTGTGTG | SEQ ID NO: 251 |
| Seq_45 | CTCGTGGGTGTACGGTAGTGTTGTTGTGTG | SEQ ID NO: 252 |
| Seq_79 | CTCGTGGGTGTACGGTTGTGTTGTTGTGTG | SEQ ID NO: 253 |
| Seq_192 | CTCGTGGGTGCACGGTGGTGTTGTTGTGTG | SEQ ID NO: 254 |
| Seq_245 | CTCGTGGGTGTACGGTGGTGCTGTTGTGTG | SEQ ID NO: 255 |

### Example 2—MRI Visualization of Hyperphosphorylated Neurons in Vivo in A P301S Mouse Model of Tau Deposition, Using Gadolinium Bearing, Thioaptamer Targeted Liposomal Nanoparticles

In vitro and ex vivo studies as described above yielded Tau_3 (SEQ ID NO: 6) as a suitable candidate thioaptamer that bound hyperphosphorylated neurons that were AT100 positive (indicative of late stage hyperphosphorylation). The inventors therefore chose to test the abilities of the Tau_3 (SEQ ID NO: 6) aptamer in targeting a payload nanoparticle to sites of tau pathology in a mouse model (P301S) of AD tauopathy. In addition, Tau_1 (SEQ ID NO: 5) targeted nanoparticles were tested because they were the most prevalent in the SELEX screen, even though they did not show binding in vitro to mouse brain tissue, in a pTau specific manner. Aptamers were synthesized with conjugatable amine terminations at the 3′ end and were linked using carbodiimide chemistry (EDC+sulfo-NHS) to carboxyl bearing liposomes (HSPC:Cholesterol:D SPE-DOTA-Gd:DSPE-PEG3400-COOH:MPEG2000DSPE:PE-Rhodamine, 31.3:40:25:0.5:3:0.2 mole ratio). More specifically, liposomes were first prepared by dissolution of the lipids and conjugates in t-butanol and hydration in saline at a total lipid concentration of 50 mM, followed by extrusion through 400 and 200 nm nucleopore track etch membranes, followed by dialysis against PBS and concentration using a hygroscopic gel to 100 mM total lipid concentration. The liposomes (5 mL) were activated with 2 mM EDC and 3 mM sulfo-NHS (corresponding to a 10× excess of EDC), followed by addition of 500 μL of aptamer (1 μmole total) at pH 7.5, and reacted for 2 hours at room temperature. After overnight storage at 4° C., the liposomes were dialyzed against PBS at pH 7.5 using a 1000 kDa cutoff, allowing free aptamer to be removed. After dialysis, the liposomes were concentrated to 1.9 mL using a 3000 Da cutoff spin column and assayed by nanodrop to quantify the aptamer concentration. It was estimated that about 400 thioaptamer molecules attached to each liposome. These procedures are described in recent publications, which are incorporated by reference herein in their entireties. Mu, Q. et al., Mol Ther Nucleic Acids 5, e382 (2016); Mann et al., Oncotarget 2, 298-304 (2011).

For MRI, P301S mice at the age of 2, 6, and 9 months, along with age matched non-transgenic siblings, were tested. This model begins to develop intracellular tau pathology at 6 months of age and has full blown intracellular and extracellular (ghost) pathology at 9 months. In this way, the inventors tested pre-pathology, pathological onset, and advanced pathology stages of the disease. Pre-scans were collected immediately prior to injection according to the following sequences: T2 weighted FSE (2 External Averages)−Scan Time: 12 min (Anatomical reference scan) TR=6500, TE=80, SliceThk=1.2 mm, Matrix=192×192, NEX=2 FA=90, Slices=16, FOV=30 mm. T1 weighted SE (4 External Averages)−Scan Time: 14 min. TR=260, TE=8.8, SliceThk=1.2 mm, Matrix=192×192, NEX=4 FA=90, Slices (2D/3D)=8/16, FOV=30 mm. T1 weighted GRE (5 Flip Angles)-Scan Time: 7 min. TR=20, TE=3.6, SliceThk=1.2 mm, Matrix=192×192, NEX=1 FA=[8 15 25 35 45 70]°, Slices=16, FOV=30 mm.

Animals were then treated with either Tau_1 (SEQ ID NO: 5) aptamer targeted liposomes, Tau_3 (SEQ ID NO: 6) aptamer targeted liposomes, or an untargeted PEGylated control liposomal preparation containing all of the remaining components (Gd chelate conjugate, rhodamine, and matrix lipids). The liposome dose was ˜250 μL per mouse, calibrated to a total of 0.2 mmol Gd per kg body weight. A brief “scout scan” confirmed contrast was aboard, following which animals were returned to their cages after recovery from anesthesia, and the contrast was allowed to circulate, extravasate, and bind to target over the course of 4 days. At the 96 hour mark (after contrast has cleared from circulation, and any remaining contrast must be bound or sequestered in some fashion), all animals were imaged using the same sequences as above, following which they were sacrificed, and the brain, liver, spleen, and kidney were harvested for follow-up analysis. Histological examination of the spleen and liver tissues showed accumulation of the contrast (visualized by the rhodamine signal), but no overt signs of toxicity.

Both Tau_1 (SEQ ID NO: 5) and Tau_3 (SEQ ID NO: 6) targeted liposomes appeared to bind to the cortex, hippocampus, and portions of the thalamus and hypothalamus in younger (2 month old) P301s transgenic animals, but not in wild type siblings (FIG. 5). Similar results were obtained at older ages as well. To quantify the accuracy of prediction by the Tau_1 (SEQ ID NO: 5) and Tau_3 (SEQ ID NO: 6) targeted liposome preparations, the signal enhancement was calculated for a 1.2 mm thick slice in a region of the brain close to Section 55 of the Paxinos atlas (including the cerebral cortex, hippocampus, and hypothalamus). The 45° GRE Sequence was used for this purpose, since it appeared to have the best signal. In separate studies, the scan-to-scan variability (95% confidence interval) in baseline signal intensity in the same animal on different days was shown to be ˜5%. Signal intensity change was therefore quantified on a 6-point scale for all 20 animals covering all 3 age groups in each comparison:

1: Definitely negative (<−10%); 2: Probably negative (−5% to −10%); 3: Possibly negative (0 to −5%); 4: Possibly positive (0 to +5%); 5: Probably positive (+5% to +10%); 6: Definitely positive (>+10%).

The JROCFIT calculator was used to calculate the ROC Curve, along with the accuracy, sensitivity, and specificity of the prediction, using genotype as the gold standard. In P301S mice, synaptic tau pathology develops beginning at 3 months of age, intracellular filaments at around 6 months, and neurofibrillary tangles at around 9 months. There is rarely any tau pathology at 2 months of age. Remarkably, even in 2-month old mice, the aptamer targeted nanoparticles showed positive signal by MRI with 80% estimated accuracy (sensitivity-57%, specificity-92%). This is an unprecedented result, in that the aptamer targeted nanoparticles can predict the onset of tau pathology in advance of intracellular tangle formation.

### Example 3—Signal Quantification from T1 Maps

Since multiple flip-angle images were acquired, calculations were performed of actual T1 values for the pre-contrast and post-contrast images. The signal equation for a spoiled gradient echo sequence is:

$$S = k [ H ] \frac{sinα ( 1 - {e}^{- TR / T 1} )}{( 1 - ( cosα ) {e}^{- TR / T 1} )} {e}^{- TE / {T 2}^{*}}$$

where k is a scaling factor and [H] is a function of spin density. Assuming constant spin density and short TE relative to T2* consistent with a T1-weighted sequence, T1 can be estimated by a non-linear fit of the signal at multiple flip angles, a well-known technique.

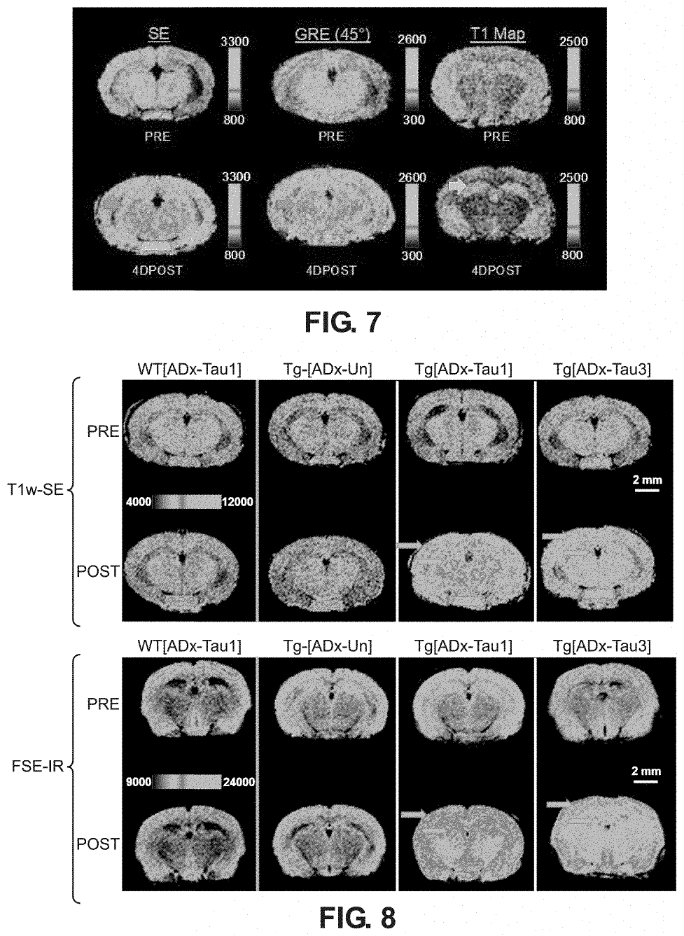

The advantage of this approach is that while T1-weighted signal intensity itself is not quantitatively relatable to contrast agent concentration, the 1/ΔT1 value is directly proportional to the concentration, with the proportionality constant equal to the molar relaxivity of the T1 shortening agent. Local contrast agent concentrations can therefore be estimated and quantify local delivery in this manner. Additionally, the T1 map is a marker of agent localization. Since the T1 map effectively takes into account information at all flip angles, it can highlight changes that are not evident at a single flip angle, as shown in FIG. 7, where the 45° flip angle image shows primarily thalamus/hypothalamus enhancement, but the T1 map additionally shows significant shortening of T1 in the hippocampus.

### Example 4—MRI Visualization of Hyperphosphorylated Neurons In Vivo in a P301S Mouse Model of Tau Deposition, Using Gadolinium Bearing, Thioaptamer Targeted Liposomal Nanoparticle s

Two liposomal formulations (“ADx-Taul” and “ADx-Tau3”) were fabricated for in vivo testing. The ADx-Taul formulation contained the amine terminated Tau-1 (SEQ ID NO: 5) aptamer (5′-/5AmMC6/CGC TCG ATA GAT CGA GCT TCG CCC ACG GTC TCC GCT CCA CAA GTT CAC GTC GAT CAC GCT CTA GAG CAC TG-3′-SEQ ID NO: 256). The ADx-Tau3 formulation contained the amine terminated Tau_3 (SEQ ID NO: 6) aptamer (5′-/5AmMC6/CGC TCG ATA GAT CGA GCT TCG CCC ACG GTC TCC GCT CCA CAA GTC CAC GTC GAT CAC GCT CTA GAG CAC TG-3′-SEQ ID NO: 257). Aptamers were synthesized with conjugatable amine terminations at the 3′ end and were linked using known carbodiimide chemistry (EDC+sulfo-NHS) to liposomes containing DSPE-PEG3400-COOH. The lipid composition and molar ratio (%) used for the fabrication of ADx-Tau formulations were HSPC:Cholesterol:DSPE-mPEG2000: DSPE-PEG3400-COOH:DSPE-DOTA-Gd=31.5:40:3:0.5:25. About 250-500 molecules of Tau_1 (SEQ ID NO: 5) aptamer and about 150-400 molecules of Tau_3 (SEQ ID NO: 6) aptamer were conjugated to the liposomes.

As a control, non-targeted ADx-Tau formulation (“ADx-Un”) lacking targeting aptamer was also fabricated (prepared without DSPE-PEG3400-COOH in lipid bilayer) and included in the in vivo study.

The efficacy of the ADx-Tau formulations was tested in a P301S mouse model of tau pathology. Animals (wild type and transgenic) underwent ADx-Tau enabled MRI at an early age (2-3 months old) for the detection of any precursor tau pathology. Histological analysis of brain sections shows fibrillar tau deposits in the cortex, hippocampus, and brain stem in ≥7 months old transgenic mice.

MRI was performed on a 1T permanent magnet scanner (M7 system, Aspect Imaging, Shoham, Israel). Mice were sedated using 2.5% isoflurane and placed on a custom fabricated bed with an integrated face-cone for continuous anesthesia delivery by inhalation (1-2% isoflurane). Respiration rate was monitored by a pneumatically controlled pressure pad placed underneath the abdomen of mice. MR images were acquired using the following sequences and scan protocols: (1) T1-weighted spin echo (Tlw-SE) sequence (Repetition Time (TR)=260 ms, Echo Time (TE)=8.5 ms, Slices=16, Voxel Size: 0.16×0.16×1.2 mm, scan time=8 min), and (2) a fast spin echo inversion recovery (FSE-IR) sequence that approximates a Tlw-fluid attenuated inversion recovery (T1w-FLAIR) sequence (TR=13500 ms, TE=86 ms, TI=2000 ms, Slices=6, Voxel Size: 0.16×0.16×2.4 mm). A 1T scanner was used because the field strength is closer to commonly used clinical 1.5 T, thus increasing translational relevance of these small animal studies, and because the relaxivity of the Gd nanoparticles is higher at low field strengths. Delayed post-contrast scans were acquired four days after intravenous administration of contrast agent (ADx-Taul, ADx-Tau3, or ADx-Un). Pre-contrast and post-contrast scans were acquired using both Tlw-SE and FSE-IR sequences with the parameters listed above. Mice were then aged to 7-9 months and euthanized for post-mortem brain histology using AT100 antibody for confirmation of pTau pathology.

To account for variability between mice and potential artifact due to positioning or MR instrument factors, the mean and standard deviation were determined for MR signal intensity for all wild type and transgenic mice for both the Tlw-SE and FSE-IR sequences. A cutoff threshold signal intensity, set as two standard deviations above the mean, was then estimated for both sequences and represented as a percentage of mean signal intensity: 5.1% (FSE-IR) and 5.6% (T1w-SE).

Qualitative and quantitative analysis of MRI images was performed in OsiriX (version 5.8.5, 64-bit) and MATLAB (version 2015a). Brain extraction was performed through a combination of threshold and manual segmentation in OsiriX. Signal change between pre-contrast and delayed post-contrast images was assessed through quantification of signal intensity in cortical regions near the center of the image stack. Tau-positive mice were identified through assessment of signal enhancement between pre-contrast and delayed post-contrast assessment of the cortex and hippocampus. The change in signal between pre-contrast and post-contrast images was quantified through integration of signal in regions of interest (ROI) that encompassed cortical tissue in central slices of the MRI volume. An observation of signal enhancement in ADx-Tau enabled delayed MR images of a tau-positive mouse (as determined by genotype and an expressed phenotype of ataxia and/or hindlimb paralysis at 7-9 months of age) above the signal variance threshold was counted as a true positive result. Conversely, signal enhancement below the signal variance threshold between pre-contrast and delayed post-contrast images for a tau-negative mouse was considered a true negative result. ROC curves were generated with a six-point ordinal scale to assess sensitivity and specificity for ADx-Tau. Sensitivity was determined by the ratio of MRI-identified true positives to the total number of true positives. Specificity was determined as the ratio of MRI-identified true negatives to the total number of true negatives. Accuracy is found as the area under the curve (AUC) of the empirical ROC curve.

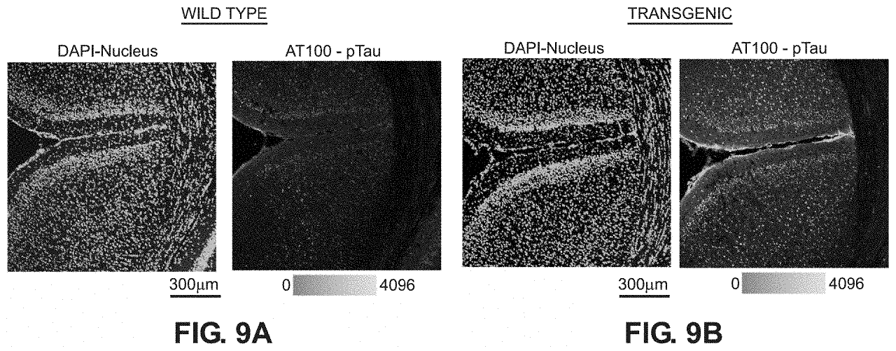

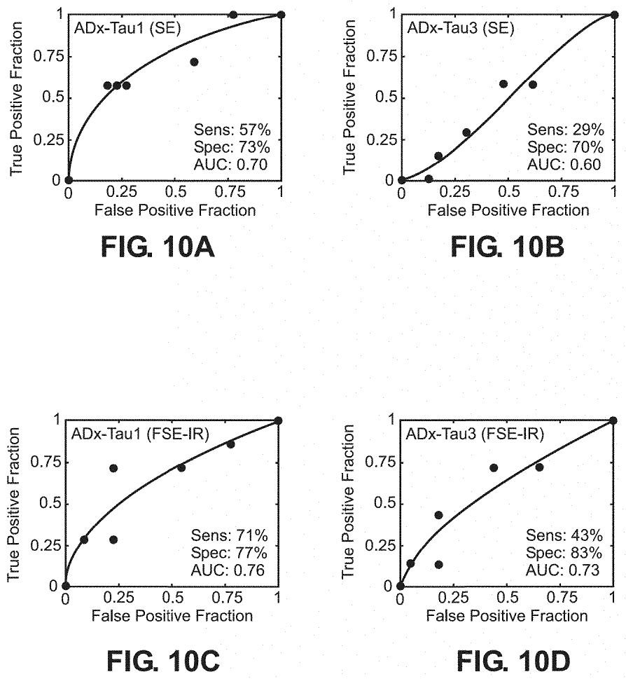

In 2-month old transgenic mice, when tau deposits have not yet occurred, there is enhancement in MR signal post injection of either the ADx-Taul or ADx-Tau3, while fewer increases occur in the wild-type mice (FIG. 8). Transgenic mice administered non-targeted formulation (ADx-Un) did not show MR signal enhancement. AT100 staining of P301S cortical brain sections demonstrated elevated pTau levels relative to wildtype counterparts (FIG. 9A-B). Comparing against genotype confirmation and expressed phenotype of ataxia and/or hindlimb paralysis at 7-9 months of age as the gold-standard, both ADx-Taul and ADx-Tau3 aptamer targeted particles yielded accuracies around 75% using the FSE-IR sequence (FIG. 10A-D).

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A composition for identifying tau pathology, comprising a targeting ligand that specifically binds to a cell surface marker of tau pathology, wherein the targeting ligand is linked to a liposome comprising an imaging agent.

2. The composition of claim 1, wherein the targeting ligand comprises an aptamer.

3. The composition of claim 1, wherein the cell surface marker of tau pathology comprises a cell surface marker of tau hyperphosphorylation.

4. The composition of claim 1, wherein the targeting ligand is determined to specifically bind to a cell surface marker of tau pathology using a systematic evolution of ligands by exponential enrichment (SELEX) method.

5. The composition of claim 1, wherein the targeting ligand comprises a DNA nucleotide sequence selected from the group consisting of Tau_1 (SEQ ID NO: 5), Tau_3 (SEQ ID NO: 6), Tau_9 (SEQ ID NO: 7), Tau_11 (SEQ ID NO: 8), Tau_10 (SEQ ID NO: 9), Tau_13 (SEQ ID NO: 10), Tau_8 (SEQ ID NO: 11), Tau_4 (SEQ ID NO: 12), Tau_17 (SEQ ID NO: 13), Tau_5 (SEQ ID NO: 14), Tau_21 (SEQ ID NO: 15), Tau_25 (SEQ ID NO: 16), Tau_7 (SEQ ID NO: 17), Tau_31 (SEQ ID NO: 18), Tau_42 (SEQ ID NO: 19), Tau_14 (SEQ ID NO: 20), Tau_19 (SEQ ID NO: 21), Tau_15 (SEQ ID NO: 22), Tau_56 (SEQ ID NO: 23), Tau_34 (SEQ ID NO: 24), Tau_23 (SEQ ID NO: 25), Tau_99 (SEQ ID NO: 26), and Tau_102 (SEQ ID NO: 27).

6. The composition of claim 5, wherein the targeting ligand comprises the DNA nucleotide sequence Tau_1 (SEQ ID NO: 5).

7. The composition of claim 5, wherein the targeting ligand comprises the DNA nucleotide sequence Tau_3 (SEQ ID NO: 6).

8. The composition of claim 1, wherein the cell surface marker of tau pathology comprises a protein selected from KRT6A, KRT6B, HSP, and VIM.

9. The composition of claim 1, wherein the imaging agent comprises a magnetic resonance imaging (MRI) contrast enhancing agent.

10. The composition of claim 1, wherein the liposome comprises a membrane, the membrane comprising:
a first phospholipid;
a sterically bulky excipient that is capable of stabilizing the liposome;
a second phospholipid that is derivatized with a first polymer;
a third phospholipid that is derivatized with a second polymer, the second polymer being conjugated to the targeting ligand; and
the imaging agent, which is encapsulated by or bound to the membrane.

11. The composition of claim 10, wherein:
the first phospholipid comprises HSPC;
the sterically bulky excipient that is capable of stabilizing the liposome comprises cholesterol;
the second phospholipid that is derivatized with a first polymer comprises DSPE-PEG;
the third phospholipid that is derivatized with a second polymer, the second polymer being conjugated to the targeting ligand comprises DSPE-PEG conjugated to at least one of Tau_1 (SEQ ID NO: 5) and Tau_3 (SEQ ID NO: 6); and
the imaging agent, which is encapsulated by or bound to the membrane comprises DSPE-DOTA-Gd.

12. The composition of claim 10, wherein:
the first phospholipid comprises HSPC;
the sterically bulky excipient that is capable of stabilizing the liposome comprises cholesterol;
the second phospholipid that is derivatized with a first polymer comprises DSPE-PEG2000;
the third phospholipid that is derivatized with a second polymer, the second polymer being conjugated to the targeting ligand comprises DSPE-PEG3400 conjugated to at least one of Tau_1 (SEQ ID NO: 5) and Tau_3 (SEQ ID NO: 6); and
the imaging agent, which is encapsulated by or bound to the membrane comprises DSPE-DOTA-Gd.

13. The composition of claim 12, wherein the ratio of HSPC: Cholesterol: DSPE-mPEG2000: DSPE-PEG3400: DSPE-DOTA-Gd=about 31.5:about 40:about 3:about 0.5:about 25.

14. The composition of claim 12, further comprising about 250-500 molecules of conjugated Tau_1 (SEQ ID NO: 5).

15. The composition of claim 12, further comprising about 150-400 molecules of conjugated Tau_3 (SEQ ID NO: 6).

16. A targeting composition, comprising a phospholipid linked to a polymer that is linked to a targeting ligand that specifically binds to a cell surface marker of tau pathology.

17. The targeting composition of claim 16, wherein the targeting ligand comprises an aptamer.

18. The targeting composition of claim 16, wherein the targeting ligand comprises a DNA nucleotide sequence selected from the group consisting of Tau_1 (SEQ ID NO: 5), Tau_3 (SEQ ID NO: 6), Tau_9 (SEQ ID NO: 7), Tau_11 (SEQ ID NO: 8), Tau_10 (SEQ ID NO: 9), Tau_13 (SEQ ID NO: 10), Tau_8 (SEQ ID NO: 11), Tau_4 (SEQ ID NO: 12), Tau_17 (SEQ ID NO: 13), Tau_5 (SEQ ID NO: 14), Tau_21 (SEQ ID NO: 15), Tau_25 (SEQ ID NO: 16), Tau_7 (SEQ ID NO: 17), Tau_31 (SEQ ID NO: 18), Tau_42 (SEQ ID NO: 19), Tau_14 (SEQ ID NO: 20), Tau_19 (SEQ ID NO: 21), Tau_15 (SEQ ID NO: 22), Tau_56 (SEQ ID NO: 23), Tau_34 (SEQ ID NO: 24), Tau_23 (SEQ ID NO: 25), Tau_99 (SEQ ID NO: 26), and Tau_102 (SEQ ID NO: 27).

19. An aptamer or stabilized aptamer comprising a DNA nucleotide sequence selected from the group consisting of Tau_1 (SEQ ID NO: 5), Tau_3 (SEQ ID NO: 6), Tau_9 (SEQ ID NO: 7), Tau_11 (SEQ ID NO: 8), Tau_10 (SEQ ID NO: 9), Tau_13 (SEQ ID NO: 10), Tau_8 (SEQ ID NO: 11), Tau_4 (SEQ ID NO: 12), Tau_17 (SEQ ID NO: 13), Tau_5 (SEQ ID NO: 14), Tau_21 (SEQ ID NO: 15), Tau_25 (SEQ ID NO: 16), Tau_7 (SEQ ID NO: 17), Tau_31 (SEQ ID NO: 18), Tau_42 (SEQ ID NO: 19), Tau_14 (SEQ ID NO: 20), Tau_19 (SEQ ID NO: 21), Tau_15 (SEQ ID NO: 22), Tau_56 (SEQ ID NO: 23), Tau_34 (SEQ ID NO: 24), Tau_23 (SEQ ID NO: 25), Tau_99 (SEQ ID NO: 26), and Tau_102 (SEQ ID NO: 27).

20. The aptamer or stabilized aptamer of claim 19, wherein the DNA nucleotide sequence is positioned between SEQ ID NO: 1 and SEQ ID NO: 2.


SEQUENCE LISTING (257 sequences)

SEQ ID NO: 1 - DNA, 23 bp, Artificial Sequence
  Synthetic
gatatgtctagagcctcagatca

SEQ ID NO: 2 - DNA, 21 bp, Artificial Sequence
  Synthetic
cggagttatgttagcagtagc

SEQ ID NO: 3 - DNA, 21 bp, Artificial Sequence
  Synthetic
cgctcgatagatcgagcttcg

SEQ ID NO: 4 - DNA, 21 bp, Artificial Sequence
  Synthetic
gtcgatcacgctctagagcac

SEQ ID NO: 5 - DNA, 30 bp, Artificial Sequence
  Synthetic
ccccccacggtctccgctccacaagttcac

SEQ ID NO: 6 - DNA, 30 bp, Artificial Sequence
  Synthetic
ccccccacggtctccgctccacaagtccac

SEQ ID NO: 7 - DNA, 30 bp, Artificial Sequence
  Synthetic
ccccccacggtctccgctccacaggttcac

SEQ ID NO: 8 - DNA, 30 bp, Artificial Sequence
  Synthetic
ctcgtgggtgtacggtggtgctgttgtgtg

SEQ ID NO: 9 - DNA, 30 bp, Artificial Sequence
  Synthetic
cccccccacggtctccgctccacaagttca

SEQ ID NO: 10 - DNA, 30 bp, Artificial Sequence
  Synthetic
ctcgtgggtgtgtggtggtgttgttgtgtg

SEQ ID NO: 11 - DNA, 30 bp, Artificial Sequence
  Synthetic
ccccccacggtctccgctccacaagcccac

SEQ ID NO: 12 - DNA, 30 bp, Artificial Sequence
  Synthetic
ctcgtcccaccacaacatcatctcaacgcc

SEQ ID NO: 13 - DNA, 30 bp, Artificial Sequence
  Synthetic
ctcgtcccaccacaacattatctcaacgcc

SEQ ID NO: 14 - DNA, 30 bp, Artificial Sequence
  Synthetic
ctcgtgggtgtacggtggtgttgttgtgtg

SEQ ID NO: 15 - DNA, 30 bp, Artificial Sequence
  Synthetic
ctccgacgggatgttcgatgagcacacact

SEQ ID NO: 16 - DNA, 30 bp, Artificial Sequence
  Synthetic
cccccccacggtctccgctccacaagtcca

SEQ ID NO: 17 - DNA, 30 bp, Artificial Sequence
  Synthetic
ccccccacggtctccgctccacaggtccac

SEQ ID NO: 18 - DNA, 30 bp, Artificial Sequence
  Synthetic
cccccattggctccgctccacacagcttca

SEQ ID NO: 19 - DNA, 30 bp, Artificial Sequence
  Synthetic
ccccccacggtctccgctccacaagctcac

SEQ ID NO: 20 - DNA, 30 bp, Artificial Sequence
  Synthetic
cccccccacggtctccgctccacaggttca

SEQ ID NO: 21 - DNA, 30 bp, Artificial Sequence
  Synthetic
ctcgtcccaccacaacattgtctcaacgcc

SEQ ID NO: 22 - DNA, 30 bp, Artificial Sequence
  Synthetic
ctcgtcccaccacaacaccatctcaacgcc

SEQ ID NO: 23 - DNA, 30 bp, Artificial Sequence
  Synthetic
ctccgacggggtgttcgatgagcacacact

SEQ ID NO: 24 - DNA, 30 bp, Artificial Sequence
  Synthetic
ccccccgcggtctccgctccacaagttcac

SEQ ID NO: 25 - DNA, 30 bp, Artificial Sequence
  Synthetic
tgggtgtgtggtggtgttgttgtgtgggtg

SEQ ID NO: 26 - DNA, 30 bp, Artificial Sequence
  Synthetic
ctcgccccaccacaacatcatctcaacgcc

SEQ ID NO: 27 - DNA, 30 bp, Artificial Sequence
  Synthetic
ccccccacggtctccgctccacaagttcgc

SEQ ID NO: 28 - DNA, 30 bp, Artificial Sequence
  Synthetic
cccccccacggtctccgctccacaagctca

SEQ ID NO: 29 - DNA, 74 bp, Artificial Sequence
  Synthetic
  misc_feature (24)..(53): n is a, c, g, or t
gatatgtctagagcctcagatcannnnnnnnnnnnnnnnnnnnnnnnnnnnnncggagttatgttagcagtagc

SEQ ID NO: 30 - DNA, 71 bp, Artificial Sequence
  Synthetic
cgctcgatagatcgagcttcgcccacggtctccgctccacaagttcacgtcgatcacgctctagagcactg

SEQ ID NO: 31 - DNA, 71 bp, Artificial Sequence
  Synthetic
cgctcgatagatcgagcttcgcccacggtctccgctccacaagtccacgtcgatcacgctctagagcactg

SEQ ID NO: 32 - DNA, 30 bp, Artificial Sequence
  Synthetic
ctttgacccaaacacaactgcggtgaatcc

SEQ ID NO: 33 - DNA, 30 bp, Artificial Sequence
  Synthetic
ctccaacctggaccccaaacgaactgagat

SEQ ID NO: 34 - DNA, 30 bp, Artificial Sequence
  Synthetic
cacgtcaaccacaccaaattggggaccgaa

SEQ ID NO: 35 - DNA, 30 bp, Artificial Sequence
  Synthetic
ctcgtacgtcaacctcaccaaattgggaac

SEQ ID NO: 36 - DNA, 30 bp, Artificial Sequence
  Synthetic
ctcgcacgtcaaccacaccaaattggggac

SEQ ID NO: 37 - DNA, 30 bp, Artificial Sequence
  Synthetic
ctcgtacgtcaaccacaccaaattggggac

SEQ ID NO: 38 - DNA, 30 bp, Artificial Sequence
  Synthetic
ctcgtctcaccacaacattatctcaacgcc

SEQ ID NO: 39 - DNA, 30 bp, Artificial Sequence
  Synthetic
ctcgtctcaccacaacatcatctcaacgcc

SEQ ID NO: 40 - DNA, 30 bp, Artificial Sequence
  Synthetic
ctcgcctcaccacaacattatctcaacgcc

SEQ ID NO: 41 - DNA, 30 bp, Artificial Sequence
  Synthetic
ctcgccccaccacaacattatctcaacgcc

SEQ ID NO: 42 - DNA, 30 bp, Artificial Sequence
  Synthetic
ctcgcctcaccacaacatcatctcaacgcc

SEQ ID NO: 43 - DNA, 30 bp, Artificial Sequence
  Synthetic
ctcgtctcaccataacattatctcaacgcc

SEQ ID NO: 44 - DNA, 30 bp, Artificial Sequence
  Synthetic
ctcgtcccaccataacattatctcaacgcc

SEQ ID NO: 45 - DNA, 30 bp, Artificial Sequence
  Synthetic
ctcgtcccaccataacatcatctcaacgcc

SEQ ID NO: 46 - DNA, 30 bp, Artificial Sequence
  Synthetic
ctcgcctcaccataacattatctcaacgcc

SEQ ID NO: 47 - DNA, 30 bp, Artificial Sequence
  Synthetic
ctcgccccaccataacattatctcaacgcc

SEQ ID NO: 48 - DNA, 30 bp, Artificial Sequence
  Synthetic
ctcgccccaccataacatcatctcaacgcc

SEQ ID NO: 49 - DNA, 30 bp, Artificial Sequence
  Synthetic
ctcgcctcaccataacatcatctcaacgcc

SEQ ID NO: 50 - DNA, 30 bp, Artificial Sequence
  Synthetic
ctcgcctcaccacaacattatcccaacgcc

SEQ ID NO: 51 - DNA, 30 bp, Artificial Sequence
  Synthetic
ctcgccccaccacaacattatcccaacgcc

SEQ ID NO: 52 - DNA, 30 bp, Artificial Sequence
  Synthetic
ctcgcctcaccacaacattgtcccaacgcc

SEQ ID NO: 53 - DNA, 30 bp, Artificial Sequence
  Synthetic
ctcgccccaccacaacattgtcccaacgcc

SEQ ID NO: 54 - DNA, 30 bp, Artificial Sequence
  Synthetic
ctcgtcccaccacaacattgtctcaatgcc

SEQ ID NO: 55 - DNA, 30 bp, Artificial Sequence
  Synthetic
ctcgccccaccacaacattgtctcaatgcc

SEQ ID NO: 56 - DNA, 30 bp, Artificial Sequence
  Synthetic
ctcgccccaccacaacattgtctcaacgcc

SEQ ID NO: 57 - DNA, 30 bp, Artificial Sequence
  Synthetic
ctcgcctcaccacaacattgtctcaacgcc

SEQ ID NO: 58 - DNA, 30 bp, Artificial Sequence
  Synthetic
ctcgtctcaccacaacattgtctcaacgcc

SEQ ID NO: 59 - DNA, 30 bp, Artificial Sequence
  Synthetic
ctcgtcccaccacaacattgcctcaacgcc

SEQ ID NO: 60 - DNA, 30 bp, Artificial Sequence
  Synthetic
ctcgtcccaccacaacatcgtctcaacgcc

SEQ ID NO: 61 - DNA, 30 bp, Artificial Sequence
  Synthetic
ctcgtcccaccataacattgtctcaacgcc

SEQ ID NO: 62 - DNA, 30 bp, Artificial Sequence
  Synthetic
ctcgtcccaccacaacactatcccaacgcc

SEQ ID NO: 63 - DNA, 30 bp, Artificial Sequence
  Synthetic
ctcgtctcaccacaacattatcccaacgcc

SEQ ID NO: 64 - DNA, 30 bp, Artificial Sequence
  Synthetic
ctcgtcccaccacaacatcatcccaacgcc

SEQ ID NO: 65 - DNA, 30 bp, Artificial Sequence
  Synthetic
ctcgtcccaccacaacattatcccaacgcc

SEQ ID NO: 66 - DNA, 30 bp, Artificial Sequence
  Synthetic
ctcgtcccaccacaacattgtcccaacgcc

SEQ ID NO: 67 - DNA, 30 bp, Artificial Sequence
  Synthetic
ctcgtcccaccacaacaatatctcaacgcc

SEQ ID NO: 68 - DNA, 30 bp, Artificial Sequence
  Synthetic
ctcgtcccaccacaacattatctcgacgcc

SEQ ID NO: 69 - DNA, 30 bp, Artificial Sequence
  Synthetic
ctcgtcccaccgcaacattatctcaacgcc

SEQ ID NO: 70 - DNA, 30 bp, Artificial Sequence
  Synthetic
ctcgcctcaccacaacattatctcaatgcc

SEQ ID NO: 71 - DNA, 30 bp, Artificial Sequence
  Synthetic
ctcgcctcaccacaacatcatctcaatgcc

SEQ ID NO: 72 - DNA, 30 bp, Artificial Sequence
  Synthetic
ctcgtcccaccacaacatcatctcaatgcc

SEQ ID NO: 73 - DNA, 30 bp, Artificial Sequence
  Synthetic
ctcgtcccaccacaacattatctcaatgcc

SEQ ID NO: 74 - DNA, 30 bp, Artificial Sequence
  Synthetic
ctcgtcccaccacaacaccatctcaatgcc

SEQ ID NO: 75 - DNA, 30 bp, Artificial Sequence
  Synthetic
ctcgtcccaccacaacactatctcaatgcc

SEQ ID NO: 76 - DNA, 30 bp, Artificial Sequence
  Synthetic
ctcgccccaccacaacaccatctcaatgcc

SEQ ID NO: 77 - DNA, 30 bp, Artificial Sequence
  Synthetic
ctcgccccaccacaacattatctcaatgcc

SEQ ID NO: 78 - DNA, 30 bp, Artificial Sequence
  Synthetic
ctcgccccaccacaacatcatctcaatgcc

SEQ ID NO: 79 - DNA, 30 bp, Artificial Sequence
  Synthetic
ctcgtcccaccacaacaccacctcaacgcc

SEQ ID NO: 80 - DNA, 30 bp, Artificial Sequence
  Synthetic
ctcgtcccaccacaacatcacctcaacgcc

SEQ ID NO: 81 - DNA, 30 bp, Artificial Sequence
  Synthetic
ctcgtcccaccataacaccatctcaacgcc

SEQ ID NO: 82 - DNA, 30 bp, Artificial Sequence
  Synthetic
ctcgtcccaccataacactatctcaacgcc

SEQ ID NO: 83 - DNA, 30 bp, Artificial Sequence
  Synthetic
ctcgtctcaccacaacactatctcaacgcc

SEQ ID NO: 84 - DNA, 30 bp, Artificial Sequence
  Synthetic
ctcgtctcaccacaacaccatctcaacgcc

SEQ ID NO: 85 - DNA, 30 bp, Artificial Sequence
  Synthetic
ctcgtcccaccacaacactatctcaacgcc

SEQ ID NO: 86 - DNA, 30 bp, Artificial Sequence
  Synthetic
ctcgtcccaccacaacactgtctcaacgcc

SEQ ID NO: 87 - DNA, 30 bp, Artificial Sequence
  Synthetic
ctcgtcccaccacaacaccgtctcaacgcc

SEQ ID NO: 88 - DNA, 30 bp, Artificial Sequence
  Synthetic
ctcgtcccaccacagcattatctcaacgcc

SEQ ID NO: 89 - DNA, 30 bp, Artificial Sequence
  Synthetic
ctcgtcccaccacagcatcatctcaacgcc

SEQ ID NO: 90 - DNA, 30 bp, Artificial Sequence
  Synthetic
ctcgtcccaccacaacattatctcagcgcc

SEQ ID NO: 91 - DNA, 30 bp, Artificial Sequence
  Synthetic
ctcgtcccaccacaacatcatctcagcgcc

SEQ ID NO: 92 - DNA, 30 bp, Artificial Sequence
  Synthetic
ctcgtcccgccacaacattatctcaacgcc

SEQ ID NO: 93 - DNA, 30 bp, Artificial Sequence
  Synthetic
ctcgtcccgccacaacatcatctcaacgcc

SEQ ID NO: 94 - DNA, 30 bp, Artificial Sequence
  Synthetic
ctcgcctcaccacaacacaatctcaatgcc

SEQ ID NO: 95 - DNA, 30 bp, Artificial Sequence
  Synthetic
ctcgcctcaccataacacaatctcaacgcc

SEQ ID NO: 96 - DNA, 30 bp, Artificial Sequence
  Synthetic
ctcgcctcaccacaacactatctcaacgcc

SEQ ID NO: 97 - DNA, 30 bp, Artificial Sequence
  Synthetic
ctcgcctcaccacaacacaatctcaacgcc

SEQ ID NO: 98 - DNA, 30 bp, Artificial Sequence
  Synthetic
ctcgcctcaccacaacaccatctcaacgcc

SEQ ID NO: 99 - DNA, 30 bp, Artificial Sequence
  Synthetic
ctcgccccaccacaacactatctcaacgcc

SEQ ID NO: 100 - DNA, 30 bp, Artificial Sequence
  Synthetic
ctcgccccaccacaacaccatctcaacgcc

SEQ ID NO: 101 - DNA, 30 bp, Artificial Sequence
  Synthetic
ctcgccccaccacaacacaatctcaacgcc

SEQ ID NO: 102 - DNA, 30 bp, Artificial Sequence
  Synthetic
ctcgccccaccacaacactgtctcaacgcc

SEQ ID NO: 103 - DNA, 30 bp, Artificial Sequence
  Synthetic
ctcgcctcaccacaacactgtctcaacgcc

SEQ ID NO: 104 - DNA, 30 bp, Artificial Sequence
  Synthetic
ctcgcctcaccacaacaccgtctcaacgcc

SEQ ID NO: 105 - DNA, 30 bp, Artificial Sequence
  Synthetic
ctcgccccaccacaacaccgtctcaacgcc

SEQ ID NO: 106 - DNA, 30 bp, Artificial Sequence
  Synthetic
ctcgtgtccacaccattcacaacgccaaat

SEQ ID NO: 107 - DNA, 30 bp, Artificial Sequence
  Synthetic
cctcaccacaacattgtctcaacgccacaa

SEQ ID NO: 108 - DNA, 30 bp, Artificial Sequence
  Synthetic
tcccaccacaacattgtctcaacgccacaa

SEQ ID NO: 109 - DNA, 30 bp, Artificial Sequence
  Synthetic
ccccaccacaacattgtctcaacgccacaa

SEQ ID NO: 110 - DNA, 30 bp, Artificial Sequence
  Synthetic
tcccaccacaacactgtctcaacgccacaa

SEQ ID NO: 111 - DNA, 30 bp, Artificial Sequence
  Synthetic
tcccaccacaacaccgtctcaacgccacaa

SEQ ID NO: 112 - DNA, 30 bp, Artificial Sequence
  Synthetic
tcccaccacaacattatctcaacgccacaa

SEQ ID NO: 113 - DNA, 30 bp, Artificial Sequence
  Synthetic
tcccaccacaacatcatctcaacgccacaa

SEQ ID NO: 114 - DNA, 30 bp, Artificial Sequence
  Synthetic
tcccaccacaacaccatctcaacgccacaa

SEQ ID NO: 115 - DNA, 30 bp, Artificial Sequence
  Synthetic
tcccaccacaacactatctcaacgccacaa

SEQ ID NO: 116 - DNA, 30 bp, Artificial Sequence
  Synthetic
tcccaccacaacattatctcaacgccacag

SEQ ID NO: 117 - DNA, 30 bp, Artificial Sequence
  Synthetic
tcccaccacaacatcatctcaacgccataa

SEQ ID NO: 118 - DNA, 30 bp, Artificial Sequence
  Synthetic
tcccaccacaacattatctcaacgccataa

SEQ ID NO: 119 - DNA, 30 bp, Artificial Sequence
  Synthetic
tcccaccacaacattgtctcaacgccataa

SEQ ID NO: 120 - DNA, 30 bp, Artificial Sequence
  Synthetic
tcccaccacaacatcatctcaatgccacaa

SEQ ID NO: 121 - DNA, 30 bp, Artificial Sequence
  Synthetic
tcccaccacaacaccatctcaatgccacaa

SEQ ID NO: 122 - DNA, 30 bp, Artificial Sequence
  Synthetic
ccccaccacaacattatctcaacgccacaa

SEQ ID NO: 123 - DNA, 30 bp, Artificial Sequence
  Synthetic
ccccaccacaacatcatctcaacgccacaa

SEQ ID NO: 124 - DNA, 30 bp, Artificial Sequence
  Synthetic
cctcaccacaacattatctcaacgccacaa

SEQ ID NO: 125 - DNA, 30 bp, Artificial Sequence
  Synthetic
cctcaccacaacatcatctcaacgccacaa

SEQ ID NO: 126 - DNA, 30 bp, Artificial Sequence
  Synthetic
ccccaccacaacaccatctcaacgccacaa

SEQ ID NO: 127 - DNA, 30 bp, Artificial Sequence
  Synthetic
cctcaccacaacacaatctcaacgccacaa

SEQ ID NO: 128 - DNA, 30 bp, Artificial Sequence
  Synthetic
gcgccgcctacgcacaaccaatcacaccat

SEQ ID NO: 129 - DNA, 30 bp, Artificial Sequence
  Synthetic
gcgccgcctacgcacaaccaatcacaccac

SEQ ID NO: 130 - DNA, 30 bp, Artificial Sequence
  Synthetic
cccacagcaccaacacacacacccgtataa

SEQ ID NO: 131 - DNA, 30 bp, Artificial Sequence
  Synthetic
cccacagcaccaacacacacatccgtataa

SEQ ID NO: 132 - DNA, 30 bp, Artificial Sequence
  Synthetic
ctcgcccacagcaccaacacacatacccgt

SEQ ID NO: 133 - DNA, 30 bp, Artificial Sequence
  Synthetic
ctcgcccacagcaccaacacacacacccgt

SEQ ID NO: 134 - DNA, 30 bp, Artificial Sequence
  Synthetic
ctcgcccacagcaccaacacacacatccgt

SEQ ID NO: 135 - DNA, 30 bp, Artificial Sequence
  Synthetic
ctcgcccacagcaccaacacacacatccgc

SEQ ID NO: 136 - DNA, 30 bp, Artificial Sequence
  Synthetic
ctcgcccacagcaccaacacacacacccgc

SEQ ID NO: 137 - DNA, 30 bp, Artificial Sequence
  Synthetic
ctccgacaccacgaggagatgcacctgcaa

SEQ ID NO: 138 - DNA, 30 bp, Artificial Sequence
  Synthetic
ctcgaacacacccagggaatacacgaaaca

SEQ ID NO: 139 - DNA, 30 bp, Artificial Sequence
  Synthetic
ctccccacagtctccaccccacaagcccac

SEQ ID NO: 140 - DNA, 30 bp, Artificial Sequence
  Synthetic
ctccccacagtctccattccacaagctcac

SEQ ID NO: 141 - DNA, 30 bp, Artificial Sequence
  Synthetic
ctccccacagtctccactccacaagctcac

SEQ ID NO: 142 - DNA, 30 bp, Artificial Sequence
  Synthetic
ctccccacagtctccattccacaggcccac

SEQ ID NO: 143 - DNA, 30 bp, Artificial Sequence
  Synthetic
ctccccacagtctccactccacaggcccac

SEQ ID NO: 144 - DNA, 30 bp, Artificial Sequence
  Synthetic
ctccccacagtctccactccacaagcccac

SEQ ID NO: 145 - DNA, 30 bp, Artificial Sequence
  Synthetic
ctccccacagtctccattccacaagcccac

SEQ ID NO: 146 - DNA, 30 bp, Artificial Sequence
  Synthetic
ctccccacagtccccactccacaagcccac

SEQ ID NO: 147 - DNA, 30 bp, Artificial Sequence
  Synthetic
ctccccacagtccccattccacaagcccac

SEQ ID NO: 148 - DNA, 30 bp, Artificial Sequence
  Synthetic
ctccccacagcctccactccacaagtccac

SEQ ID NO: 149 - DNA, 30 bp, Artificial Sequence
  Synthetic
ctccccacagcctccactccacaagcccac

SEQ ID NO: 150 - DNA, 30 bp, Artificial Sequence
  Synthetic
ctccccacagcctccattccacaagctcac

SEQ ID NO: 151 - DNA, 30 bp, Artificial Sequence
  Synthetic
ctccccacagcctccattccacaagcccac

SEQ ID NO: 152 - DNA, 30 bp, Artificial Sequence
  Synthetic
ctccccacagcccccattccacaagcccac

SEQ ID NO: 153 - DNA, 30 bp, Artificial Sequence
  Synthetic
ctccccacagtctccactccacaagttcac

SEQ ID NO: 154 - DNA, 30 bp, Artificial Sequence
  Synthetic
ctccccacagtccccactccacaagttcac

SEQ ID NO: 155 - DNA, 30 bp, Artificial Sequence
  Synthetic
ctccccacagtctccattccacaagttcac

SEQ ID NO: 156 - DNA, 30 bp, Artificial Sequence
  Synthetic
ctccccacagtccccattccacaagttcac

SEQ ID NO: 157 - DNA, 30 bp, Artificial Sequence
  Synthetic
ctccccacagcctccattccacaagttcac

SEQ ID NO: 158 - DNA, 30 bp, Artificial Sequence
  Synthetic
ctccccacagcctccactccacaagttcac

SEQ ID NO: 159 - DNA, 30 bp, Artificial Sequence
  Synthetic
ctccccacagcccccactccacaagttcac

SEQ ID NO: 160 - DNA, 30 bp, Artificial Sequence
  Synthetic
ctccccacagcccccattccacaagttcac

SEQ ID NO: 161 - DNA, 30 bp, Artificial Sequence
  Synthetic
ctccccacagcccccactccacaagtccac

SEQ ID NO: 162 - DNA, 30 bp, Artificial Sequence
  Synthetic
ctccccacagtctccactccacaagtccac

SEQ ID NO: 163 - DNA, 30 bp, Artificial Sequence
  Synthetic
ctccccacagtccccactccacaagtccac

SEQ ID NO: 164 - DNA, 30 bp, Artificial Sequence
  Synthetic
ctccccacagcctccattccacaagtccac

SEQ ID NO: 165 - DNA, 30 bp, Artificial Sequence
  Synthetic
ctccccacagtctccattccacaagtccac

SEQ ID NO: 166 - DNA, 30 bp, Artificial Sequence
  Synthetic
ctccccacagtccccattccacaagtccac

SEQ ID NO: 167 - DNA, 30 bp, Artificial Sequence
  Synthetic
ctccccacagcccccattccacaagtccac

SEQ ID NO: 168 - DNA, 30 bp, Artificial Sequence
  Synthetic
ctccccacagtctccaccccacaagttcac

SEQ ID NO: 169 - DNA, 30 bp, Artificial Sequence
  Synthetic
ctccccacagcctccaccccacaagttcac

SEQ ID NO: 170 - DNA, 30 bp, Artificial Sequence
  Synthetic
ctccccacagtccccactccacaggttcac

SEQ ID NO: 171 - DNA, 30 bp, Artificial Sequence
  Synthetic
ctccccacagtctccactccacaggttcac

SEQ ID NO: 172 - DNA, 30 bp, Artificial Sequence
  Synthetic
ctccccacagtctccattccacaggttcac

SEQ ID NO: 173 - DNA, 30 bp, Artificial Sequence
  Synthetic
ctccccacagcctccattccacaggttcac

SEQ ID NO: 174 - DNA, 30 bp, Artificial Sequence
  Synthetic
ctccccacagcctccactccacaggttcac

SEQ ID NO: 175 - DNA, 30 bp, Artificial Sequence
  Synthetic
ccccccattggctccgctccacacagcttc

SEQ ID NO: 176 - DNA, 30 bp, Artificial Sequence
  Synthetic
cccccattggctccgctccacacggcttca

SEQ ID NO: 177 - DNA, 30 bp, Artificial Sequence
  Synthetic
cccccattggctccgctccacacaacttca

SEQ ID NO: 178 - DNA, 30 bp, Artificial Sequence
  Synthetic
cccccattggctccgctccacacagcctca

SEQ ID NO: 179 - DNA, 30 bp, Artificial Sequence
  Synthetic
cccccccgcggtctccgctccacaagttca

SEQ ID NO: 180 - DNA, 30 bp, Artificial Sequence
  Synthetic
cccccccacggtctccgctccacaagccca

SEQ ID NO: 181 - DNA, 30 bp, Artificial Sequence
  Synthetic
cccccccacggtctccgctccacaggtcca

SEQ ID NO: 182 - DNA, 30 bp, Artificial Sequence
  Synthetic
cccccacggtctccgctccacaagttcaca

SEQ ID NO: 183 - DNA, 30 bp, Artificial Sequence
  Synthetic
cccccacggtctccgctccacaagtccaca

SEQ ID NO: 184 - DNA, 30 bp, Artificial Sequence
  Synthetic
ccccccacggtctccgctccacaagcacac

SEQ ID NO: 185 - DNA, 30 bp, Artificial Sequence
  Synthetic
ccccccacggtctccgctccacaggcccac

SEQ ID NO: 186 - DNA, 30 bp, Artificial Sequence
  Synthetic
ccccccacggtctccgctccacaggctcac

SEQ ID NO: 187 - DNA, 30 bp, Artificial Sequence
  Synthetic
ccccccacggtctccgctccacaagtccgc

SEQ ID NO: 188 - DNA, 30 bp, Artificial Sequence
  Synthetic
ccccccacggcctccgctccacaagttcac

SEQ ID NO: 189 - DNA, 30 bp, Artificial Sequence
  Synthetic
ccccccacggcctccgctccacaagtccac

SEQ ID NO: 190 - DNA, 30 bp, Artificial Sequence
  Synthetic
ccccccccggtctccgctccacaagttcac

SEQ ID NO: 191 - DNA, 30 bp, Artificial Sequence
  Synthetic
ccccccgcggtctccgctccacaagtccac

SEQ ID NO: 192 - DNA, 30 bp, Artificial Sequence
  Synthetic
ctctctggtccccccggccgtccctctcat

SEQ ID NO: 193 - DNA, 30 bp, Artificial Sequence
  Synthetic
ctcgtaccacccccggccgtccctctcatc

SEQ ID NO: 194 - DNA, 30 bp, Artificial Sequence
  Synthetic
ctccccgtccacctcgcacccaaggcaatc

SEQ ID NO: 195 - DNA, 30 bp, Artificial Sequence
  Synthetic
ctccccgtccacctcgcactcaaggcaatc

SEQ ID NO: 196 - DNA, 30 bp, Artificial Sequence
  Synthetic
ctccccgtccaccccgcactcaaggcaatc

SEQ ID NO: 197 - DNA, 30 bp, Artificial Sequence
  Synthetic
ctcccccccacctggcactgtccccggaga

SEQ ID NO: 198 - DNA, 30 bp, Artificial Sequence
  Synthetic
ctccccacctggcactgtcccaacgccaca

SEQ ID NO: 199 - DNA, 30 bp, Artificial Sequence
  Synthetic
ctcggccagcagttacagcacaccacactt

SEQ ID NO: 200 - DNA, 30 bp, Artificial Sequence
  Synthetic
ctccgacgggatgttcgacgagcacacact

SEQ ID NO: 201 - DNA, 30 bp, Artificial Sequence
  Synthetic
ctccgacggggtgttcgacgagcacacact

SEQ ID NO: 202 - DNA, 30 bp, Artificial Sequence
  Synthetic
ccccgacgggatgttcgatgagcacacact

SEQ ID NO: 203 - DNA, 30 bp, Artificial Sequence
  Synthetic
ctccgacgggatgttcgatgagcacacacc

SEQ ID NO: 204 - DNA, 31 bp, Artificial Sequence
  Synthetic
tgccctccgctcgtattgtcaccccgcaatg

SEQ ID NO: 205 - DNA, 30 bp, Artificial Sequence
  Synthetic
cgcctgctgccttcccatacgtcgatccag

SEQ ID NO: 206 - DNA, 30 bp, Artificial Sequence
  Synthetic
cgcctgctgccttcccacacgtcgatccag

SEQ ID NO: 207 - DNA, 30 bp, Artificial Sequence
  Synthetic
cgcctgctgccttcctatacgccgatccag

SEQ ID NO: 208 - DNA, 30 bp, Artificial Sequence
  Synthetic
cgcctgctgccttcctatacgtcgatccag

SEQ ID NO: 209 - DNA, 30 bp, Artificial Sequence
  Synthetic
cgcctgctgccttcctgtacgtcgatccag

SEQ ID NO: 210 - DNA, 30 bp, Artificial Sequence
  Synthetic
cgcctgctgccttcctgtacgccgatccag

SEQ ID NO: 211 - DNA, 30 bp, Artificial Sequence
  Synthetic
ctcgctgaccagatgaggggggtttactgg

SEQ ID NO: 212 - DNA, 30 bp, Artificial Sequence
  Synthetic
ctcgctgaccagatgaagggggtttactgg

SEQ ID NO: 213 - DNA, 30 bp, Artificial Sequence
  Synthetic
ctcgccgaccagatgaaggggggtttactg

SEQ ID NO: 214 - DNA, 30 bp, Artificial Sequence
  Synthetic
ctcgctgaccagatggaggggggtttactg

SEQ ID NO: 215 - DNA, 30 bp, Artificial Sequence
  Synthetic
ctcgctgaccaggtgaaggggggtttactg

SEQ ID NO: 216 - DNA, 30 bp, Artificial Sequence
  Synthetic
ctcgctggccagatgaaggggggtttactg

SEQ ID NO: 217 - DNA, 30 bp, Artificial Sequence
  Synthetic
ctcgctgaccggatgaaggggggtttactg

SEQ ID NO: 218 - DNA, 30 bp, Artificial Sequence
  Synthetic
ctcgctgaccagacgaaggggggtttactg

SEQ ID NO: 219 - DNA, 30 bp, Artificial Sequence
  Synthetic
ctcgctgaccagatgaaggggggtttgctg

SEQ ID NO: 220 - DNA, 30 bp, Artificial Sequence
  Synthetic
ctcgctgaccagatgagggggggtttactg

SEQ ID NO: 221 - DNA, 30 bp, Artificial Sequence
  Synthetic
ctcgctgaccagatgaaggggggtttactg

SEQ ID NO: 222 - DNA, 30 bp, Artificial Sequence
  Synthetic
ctcgctgaccagatgaaggggggcttactg

SEQ ID NO: 223 - DNA, 30 bp, Artificial Sequence
  Synthetic
ctgaccagatgaagggggggtttactgggg

SEQ ID NO: 224 - DNA, 30 bp, Artificial Sequence
  Synthetic
ctgaccagatggaggggggtttactggggg

SEQ ID NO: 225 - DNA, 30 bp, Artificial Sequence
  Synthetic
ccgaccagatgaaggggggtttactggggg

SEQ ID NO: 226 - DNA, 30 bp, Artificial Sequence
  Synthetic
ctggccagatgaaggggggtttactggggg

SEQ ID NO: 227 - DNA, 30 bp, Artificial Sequence
  Synthetic
ctgaccaggtgaaggggggtttactggggg

SEQ ID NO: 228 - DNA, 30 bp, Artificial Sequence
  Synthetic
ctgaccggatgaaggggggtttactggggg

SEQ ID NO: 229 - DNA, 30 bp, Artificial Sequence
  Synthetic
ctgaccagatgagggggggtttactggggg

SEQ ID NO: 230 - DNA, 30 bp, Artificial Sequence
  Synthetic
ctgaccagatgaaggggggtttgctggggg

SEQ ID NO: 231 - DNA, 30 bp, Artificial Sequence
  Synthetic
ctgaccagatgaaggggggtttactggggg

SEQ ID NO: 232 - DNA, 30 bp, Artificial Sequence
  Synthetic
ctgaccagatgaaggggggcttactggggg

SEQ ID NO: 233 - DNA, 30 bp, Artificial Sequence
  Synthetic
gtgggtgtgtatgtgtggcgggggtgcgtt

SEQ ID NO: 234 - DNA, 30 bp, Artificial Sequence
  Synthetic
ggtgtattctccgtggcgggggtgcgttgg

SEQ ID NO: 235 - DNA, 30 bp, Artificial Sequence
  Synthetic
ttgggtgtattctccgtggcggggtgcgtt

SEQ ID NO: 236 - DNA, 30 bp, Artificial Sequence
  Synthetic
ctcgggttcatgtgttgtgtgggtgggggt

SEQ ID NO: 237 - DNA, 30 bp, Artificial Sequence
  Synthetic
ggttcatgtgttgtgtgggtgggggtgtgt

SEQ ID NO: 238 - DNA, 30 bp, Artificial Sequence
  Synthetic
ctcggtgtccagattgatgttggggtgggg

SEQ ID NO: 239 - DNA, 30 bp, Artificial Sequence
  Synthetic
tgggtgtgcggtggtgttgttgtgtgggtg

SEQ ID NO: 240 - DNA, 30 bp, Artificial Sequence
  Synthetic
tgggtgtgtggtggtgttgttgtgtggatg

SEQ ID NO: 241 - DNA, 30 bp, Artificial Sequence
  Synthetic
tgggtgtacggtggtgttgttgtgtgggtg

SEQ ID NO: 242 - DNA, 30 bp, Artificial Sequence
  Synthetic
tgggtatacggtggtgttgttgtgtgggtg

SEQ ID NO: 243 - DNA, 30 bp, Artificial Sequence
  Synthetic
tgggtgtacggtagtgttgttgtgtgggtg

SEQ ID NO: 244 - DNA, 30 bp, Artificial Sequence
  Synthetic
tgggtgtacggttgtgttgttgtgtgggtg

SEQ ID NO: 245 - DNA, 30 bp, Artificial Sequence
  Synthetic
ctcgtgggtatgcggtggtgttgttgtgtg

SEQ ID NO: 246 - DNA, 30 bp, Artificial Sequence
  Synthetic
ctcgtgggtgtatggtggtgttgttgtgtg

SEQ ID NO: 247 - DNA, 30 bp, Artificial Sequence
  Synthetic
ctcgcgggtgtgtggtggtgttgttgtgtg

SEQ ID NO: 248 - DNA, 30 bp, Artificial Sequence
  Synthetic
ctcgtgggtgtgtggtagtgttgttgtgtg

SEQ ID NO: 249 - DNA, 30 bp, Artificial Sequence
  Synthetic
ctcgtgggtgtgtggttgtgttgttgtgtg

SEQ ID NO: 250 - DNA, 30 bp, Artificial Sequence
  Synthetic
ctcgtgggtgtgtggtggtgctgttgtgtg

SEQ ID NO: 251 - DNA, 30 bp, Artificial Sequence
  Synthetic
ctcgtgggtgtgcggtggtgttgttgtgtg

SEQ ID NO: 252 - DNA, 30 bp, Artificial Sequence
  Synthetic
ctcgtgggtatacggtagtgttgttgtgtg

SEQ ID NO: 253 - DNA, 30 bp, Artificial Sequence
  Synthetic
ctcgtgggtatacggttgtgttgttgtgtg

SEQ ID NO: 254 - DNA, 30 bp, Artificial Sequence
  Synthetic
ctcgtgggtatacggtggtgttgttgtgtg

SEQ ID NO: 255 - DNA, 30 bp, Artificial Sequence
  Synthetic
ctcgtgggtgtacggtagtgttgttgtgtg

SEQ ID NO: 256 - DNA, 30 bp, Artificial Sequence
  Synthetic
ctcgtgggtgtacggttgtgttgttgtgtg

SEQ ID NO: 257 - DNA, 30 bp, Artificial Sequence
  Synthetic
ctcgtgggtgcacggtggtgttgttgtgtg